(19) United States
(12) Patent Application Publication
(10) Pub. No.: US 2024/0374158 A1
(43) Pub. Date: Nov. 14, 2024

(54) FLUID STATUS DETERMINATION USING BIOIMPEDANCE

(71) Applicant: Fresenius Medical Care Holdings, Inc., Waltham, MA (US)

(72) Inventors: Fansan Zhu, Flushing, NY (US); Peter Kotanko, New York, NY (US)

(21) Appl. No.: 18/653,801

(22) Filed: May 2, 2024

Related U.S. Application Data
(60) Provisional application No. 63/501,478, filed on May 11, 2023.

(51) Int. Cl. A61B 5/0537

(52) U.S. Cl. CPC A61B 5/0537

(57) ABSTRACT

The present teachings generally include devices, systems, methods, and computer-program products for determining a fluid status of patients using bioimpedance measurements and the like. In general, aspects of the present teachings may include one or more of: an eight-point electrode bioimpedance device including two (wired or wireless) handles with integrated sensors to measure distance, e.g., so that body height and segmental length can be measured; techniques to determine the fluid status of a patient from bioimpedance measurements of the patient's legs; improved techniques for determining fluid overload (or otherwise determining fluid status) using a ratio of extracellular volume (ECV) to total body water (TBW); and using body segmental resistances to determine fluid status and/or fluid volume.

### CROSS-REFERENCE TO RELATED APPLICATIONS

This application claims priority to U.S. Provisional Pat. App. No. 63/501,478 filed on May 11, 2023, the entire contents of which are hereby incorporated by reference.

### FIELD

The present disclosure generally relates to techniques for determining a fluid status of patients using bioimpedance measurements and the like.

### BACKGROUND

Measurement of fluid status can be challenging in chronic kidney disease (CKD) and dialysis patients. Currently, whole body bioimpedance can be applied for assessing overhydration in clinical studies. However, the accuracy of measurements by whole body methods may be affected by change in variability of fluid distribution between body segments in the patients (see, e.g., Sommerer C., et al., “Bioimpedance analysis is not superior to clinical assessment in determining hydration status: A prospective randomized-controlled trial in a Western dialysis population,” Hemodial. Int. (2021) 10.1111/hdi.12919, which is hereby incorporated by reference herein). For example, it was found that the degree of hydration in the legs in hemodialysis (HD) patients is higher than in other body segments (e.g., arms, torso). Correspondingly, an approach of using calf bioimpedance was proposed for measuring hydration state (see Zhu F., et al., “Estimation of normal hydration in dialysis patients using whole body and calf bioimpedance analysis,” Physiol. Meas., 32: 887-902 (2011) 10.1088/0967-3334/32/7/S12, which is hereby incorporated by reference herein). However, improved techniques for measuring fluid status are desired.

Estimation of overhydration (OH) can be a major challenge in patients with CKD and end stage renal disease (ESRD). Bioimpedance has been considered a useful tool in clinical practice. Two kinds of bioimpedance devices are generally commercially available to estimate fluid status in clinical study—a Body Composition Monitor (BCM), produced by Fresenius Medical Care AG & Co. KGaA, with four skin electrodes placed on the hand, wrist, ankle, and foot (four-point electrodes method) to provide the value of OH (kg) in dialysis patients; and others that use eight-point metal electrodes instead of skin electrodes. With eight-point electrode devices, four electrodes are built in a footplate, while another four electrodes are built on two handles so that measurements will be automatically performed when a subject stands on the footplate and grips the handles, where eight-point bioimpedance devices may not require skin electrodes. This can be advantageous as it can reduce the cost of electrodes but also can simplify the procedure and save operational time, e.g., compared to a BCM. However, a major limitation of eight-point devices is that they generally cannot provide a quantitative value of OH. Instead, they typically report a ratio of extracellular volume (ECV) to total body water (TBW), the ratio being ECV/TBW, to estimate the body's hydration according to a normal range. However, it is difficult to apply ECV/TBW in a clinical routine because the normal range is often too wide. Moreover, the ratio of ECV/TBW should be converted to a value of fluid volume (kg) for convenience in clinical practice. In addition, both four-point and eight-point electrodes bioimpedance devices may require a manual input of the subject's height, where errors can significantly affect the accuracy.

There remains a need for improved techniques for determining a fluid status of patients using bioimpedance measurements and the like.

### SUMMARY

The present teachings generally include devices, systems, methods, and computer-program products for determining a fluid status of patients using bioimpedance measurements and the like. In general, aspects of the present teachings may include one or more of: an eight-point electrode bioimpedance device including two (wired or wireless) handles with integrated sensors to measure distance, e.g., so that body height and segmental length can be measured; techniques to determine the fluid status of a patient from bioimpedance measurements of the patient's legs; improved techniques for determining fluid overload (or otherwise determining fluid status) using a ratio of extracellular volume (ECV) to total body water (TBW); and using body segmental resistances to determine fluid status and/or fluid volume.

In an aspect, a method of determining fluid status of a patient disclosed herein may include: receiving a first foot of the patient on a footplate of a bioimpedance device, the footplate including a first foot electrode positioned for electrical contact with a forefoot portion of the first foot and a second foot electrode positioned for electrical contact with a hindfoot portion of the first foot; receiving a second foot of the patient on the footplate, the footplate including a third foot electrode positioned for electrical contact with a forefoot portion of the second foot and a fourth foot electrode positioned for electrical contact with a hindfoot portion of the second foot; measuring a leg resistance, R_{L}, of the patient using the footplate by measuring resistance to a current passing through each of the first foot electrode, the second foot electrode, the third foot electrode, and the fourth foot electrode; and providing the measured leg resistance, R_{L}, to a computing device including a processor and a memory. The memory may store code executable by the processor to: calculate a hydration factor, α_{P}, by dividing the measured leg resistance, R_{L}, by height of the patient, H; calculate a hydration factor delta, Aa, by subtracting the hydration factor, α_{P}, from a given baseline hydration factor, α_{N}, the given baseline hydration factor related to a plurality of individuals; and calculate a fluid overload in legs of the patient, FO_{L}, by multiplying the hydration factor delta, Δα, by a fluid volume in legs of the patient, V_{L}. Other embodiments of this aspect include corresponding computer systems, apparatus, and computer programs recorded on one or more computer storage devices, each configured to perform one or more of the aforementioned steps.

Implementations may include one or more of the following features. The current may have a frequency less than or equal to 5 kilohertz. The computing device may be included on the bioimpedance device. The computing device may be included on the footplate. The computing device may be an external device in communication with the bioimpedance device. The fluid volume in legs of the patient, V_{L}, may be determined using the equation V_{L}=C_{L}*H/R_{L}, where C_{L} (Ω*cm²) is a constant coefficient. The constant coefficient, C_{L}, may be obtained by analyzing a plurality of subjects each having a known fluid volume in legs thereof, a known height, and a measured leg resistance. Leg resistance of the plurality of subjects may be measured using a frequency less than or equal to 5 kilohertz. The fluid volume in legs of the patient, V_{L}, may be determined using the equation V_{L}=H*(a+C_{L}/R_{L})−b, where C_{L} (Ω*cm²) is a constant coefficient, and constants a and b are determined from subjects to account for error as a function of H. The calculation of the fluid overload in legs of the patient, FO_{L}, may be further refined by multiplying Δα by V_{L} and a coefficient constant, c. The coefficient constant, c, may be determined by a calibration value from a ratio (Q/cm). The memory may store code executable by the processor to calculate whole body fluid overload, FO_{W}, using the equation, FO_{W}=FO_{N}+FO_{L}+λ*BMI, where FO_{N} is a baseline fluid status, FO_{L} is the calculated fluid overload in legs of the patient, λ is a constant coefficient (L*m²/kg) related to the patient, and BMI is a measured body mass index (kg/m²) for the patient. FO_{N} may be determined by a model fit to data from patients considered to be healthy, λ may be determined by a model fit to data from patients considered to be healthy. BMI may be provided using sensors of the footplate. The height of the patient may be input provided by a user. The user may be the patient. The height of the patient may be determined, at least in part, by one or more sensors in communication with one or more of the footplate and the computing device. Implementations of the described techniques may include hardware, a method or process, or computer software on a computer-accessible medium.

In an aspect, a bioimpedance device may include: a plurality of handles including a first handle and a second handle, where each of the first handle and the second handle include at least two electrodes configured to measure bioimpedance of a patient holding the plurality of handles; a footplate including at least four electrodes configured to measure bioimpedance of the patient standing on the footplate, the footplate electrically couplable to each of the plurality of handles; and one or more sensors disposed on at least one of the plurality of handles, the one or more sensors configured to determine a height of the patient.

Implementations may include one or more of the following features. The height of the patient may be determined by: the patient gripping the first handle in a first hand and the second handle in a second hand; the patient moving each of the first handle and the second handle from a first elevation disposed below a top of the head of the patient to a second elevation disposed immediately adjacent to the top of the head of the patient; the one or more sensors indicating when at least one of the first handle and the second handle is disposed at the second elevation; and measuring height of the second elevation relative to a surface using the one or more sensors. The surface may be a ground surface and the patient may be standing on the footplate that is positioned on the ground surface, and the height of the patient may further be determined by subtracting a height of the footplate from the height of the second elevation relative to the ground surface. At least one of the one or more sensors may be an optical sensor. One or more of the plurality of handles may include circuitry configured for wireless communication with one or more of the footplate, another handle of the plurality of handles, and a computing device. Each of the plurality of handles may include a connection port structurally configured to receive a wire for passing a current therethrough during a bioimpedance measurement. The connection port may include a magnet. The one or more sensors may be configured to provide data to determine a body segmental length of the patient. The body segmental length of the patient may be determined by: the patient gripping the first handle in a first hand and the second handle in a second hand; the patient extending each of the first hand and the second hand from sides of the patient at a furthest position possible from a torso of the patient; and measuring, using the one or more sensors, a distance between the first handle and the second handle, the distance representing the body segmental length. The first handle and the second handle may be configured to measure a resistance across the body segmental length of the patient. The resistance may be provided to a processor configured by code stored in a memory to calculate resistance in each arm of the patient. The calculated resistance may assume that resistances in each arm of the patient are equal, and that resistance across the torso of the patient is negligible.

In an aspect, a computer program product for providing fluid status information for patients disclosed herein may include computer executable code embodied in a non-transitory computer readable medium that, when executing on one or more computing devices, performs the steps of: receiving a ratio (ECV/TBW) of extracellular volume, ECV, to total body water, TBW, the ratio provided by a body composition model using input from a bioimpedance device having at least eight electrodes; receiving a plurality of coefficients determined using a multiple regression analysis, the plurality of coefficients including constants β, γ, and δ, where β is a baseline fluid overload determined by analyzing a plurality of subjects, γ is a constant related to body mass index, and δ is a constant related to the ratio ECV/TBW; and calculating fluid overload (FO_{BCM}) for a patient using the formula FO_{BCM}=β+γ*BMI+δ*(ECV/TBW). Other embodiments of this aspect include corresponding computer systems, apparatus, methods, and computer programs recorded on one or more computer storage devices, each configured to perform one or more of the aforementioned steps.

In an aspect, a method disclosed herein may include: receiving a first foot of a patient on a footplate of a bioimpedance device, the footplate including a first foot electrode positioned for electrical contact with a forefoot portion of the first foot and a second foot electrode positioned for electrical contact with a hindfoot portion of the first foot; receiving a second foot of the patient on the footplate, the footplate including a third foot electrode positioned for electrical contact with a forefoot portion of the second foot and a fourth foot electrode positioned for electrical contact with a hindfoot portion of the second foot; receiving a first hand of the patient gripping a first handle of the bioimpedance device, the first handle including a first hand electrode positioned for electrical contact with a first portion of the first hand and a second hand electrode positioned for electrical contact with a second portion of the first hand; receiving a second hand of the patient gripping a second handle of the bioimpedance device, the second handle including a third hand electrode positioned for electrical contact with a first portion of the second hand and a fourth hand electrode positioned for electrical contact with a second portion of the second hand; with the patient fully extending their arms away from their body, measuring a resistance, R_{A}, across a body segmental length of the patient; with the first foot electrode electrically coupled to the first hand electrode and with the second foot electrode electrically coupled to the second hand electrode, measuring a resistance, R_{W1}, across a first side of the patient; with the third foot electrode electrically coupled to the third hand electrode and with the fourth foot electrode electrically coupled to the fourth hand electrode, measuring a resistance, R_{W2}, across a second side of the patient; with the third foot electrode electrically coupled to the first hand electrode and with the fourth foot electrode electrically coupled to the second hand electrode, measuring a resistance, R_{W12}, across the patient; with the first foot electrode electrically coupled to the third hand electrode and with the second foot electrode electrically coupled to the fourth hand electrode, measuring a resistance, R_{W21}, across the patient; and providing R_{A}, R_{W1}, R_{W2}, R_{W12}, and R_{W21} to a computing device including a processor and a memory. The memory may store code executable by the processor to calculate values for unknown variables by solving simultaneous linear equations, including: R_{W1}=R_{A1}+R_{T1}+R_{L1}, where R_{A1} represents a first arm resistance, R_{T1} represents a first side trunk resistance, and R_{L1} represents a first leg resistance; R_{W2}=R_{A2}+R_{T2}+R_{L2}, where R_{A2} represents a second arm resistance, R_{T2} represents a second side trunk resistance, and R_{L2} represents a second leg resistance; R_{W12}=R_{A1}+R_{T12}+R_{L2}, where R_{T12} represents a resistance from an upper first side to a lower second side of a trunk of the patient; and R_{W21}=R_{A2}+R_{T21}+R_{L1}, where R_{T21} represents a resistance from an upper second side to a lower first side of the trunk of the patient, where R_{A}/2=R_{A1}=R_{A2}; R_{T1}=R_{T2}; and R_{T12}=R_{T21}. Other embodiments of this aspect include corresponding computer systems, apparatus, and computer programs recorded on one or more computer storage devices, each configured to perform one or more of the aforementioned steps.

Implementations may include one or more of the following features. The method may further include calculating, via the processor, extracellular volume (ECV) in the specific body segment, i, of the patient using the formula ECVᵢ=C_{iE}*H/R_{iE}, C_{iE} is an extracellular coefficient constant in segment i, H is the height of the patient, and R_{iE} is extracellular resistance measured in the segment i by the bioimpedance device. The method may further include calculating, via the processor, intracellular volume (ICV) in the specific body segment, i, of the patient using the formula ICVᵢ=CG_{iI}*H/R_{iI}, C_{iI} is an intracellular coefficient constant in segment i, H is the height of the patient, and R_{iI} is intracellular resistance measured in the segment i by the bioimpedance device. Implementations of the described techniques may include hardware, a method or process, or computer software on a computer-accessible medium.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a patient on a footplate of a bioimpedance device in relation to a method of determining fluid status of the patient, in accordance with a representative embodiment.

FIG. 2 is a schematic representation of resistances of a patient in relation to a method of determining fluid status of the patient, in accordance with a representative embodiment.

FIG. 3 shows a patient positioned for use of a bioimpedance device, in accordance with a representative embodiment.

FIG. 4 shows handles and a footplate of a bioimpedance device, in accordance with a representative embodiment.

FIG. 5 shows a perspective view of a handle, in accordance with a representative embodiment.

FIG. 6 shows a bottom plan view of a handle, in accordance with a representative embodiment.

FIG. 7 shows a patient using a bioimpedance device, in accordance with a representative embodiment.

FIG. 8 is a schematic representation of body segmental length of a patient and a resistance thereof, in accordance with a representative embodiment.

FIG. 9A is a schematic diagram relating to a method of determining whole body bioimpedance measurement from a right side, in accordance with a representative embodiment.

FIG. 9B is a schematic diagram relating to a method of determining whole body bioimpedance measurement from a left side, in accordance with a representative embodiment.

FIG. 10A is a schematic diagram relating to a method of determining whole body bioimpedance measurement between a right arm and a left leg, in accordance with a representative embodiment.

FIG. 10B is a schematic diagram relating to a method of determining whole body bioimpedance measurement between a left arm and a right leg, respectively, in accordance with a representative embodiment.

FIG. 11A is a plot of calculated extracellular volume (ECV) vs. measured ECV of an arm of a patient, in accordance with a representative embodiment.

FIG. 11B is a Bland-Altman plot comparing calculated ECV and measured ECV of an arm of a patient, in accordance with a representative embodiment.

FIG. 11C is a plot of calculated ECV vs. measured ECV of a trunk of a patient, in accordance with a representative embodiment.

FIG. 11D is a Bland-Altman plot comparing calculated ECV and measured ECV of a trunk of a patient, in accordance with a representative embodiment.

FIG. 11E is a plot of calculated ECV vs. measured ECV of a leg of a patient, in accordance with a representative embodiment.

FIG. 11F is a Bland-Altman plot comparing calculated ECV and measured ECV of a leg of a patient, in accordance with a representative embodiment.

FIG. 12A is a plot of calculated total body water (TBW) vs. measured TBW of an arm of a patient, in accordance with a representative embodiment.

FIG. 12B is a Bland-Altman plot comparing calculated TBW and measured TBW of an arm of a patient, in accordance with a representative embodiment.

FIG. 12C is a plot of calculated TBW vs. measured TBW of a trunk of a patient, in accordance with a representative embodiment.

FIG. 12D is a Bland-Altman plot comparing calculated TBW and measured TBW of a trunk of a patient, in accordance with a representative embodiment.

FIG. 12E is a plot of calculated TBW vs. measured TBW of a leg of a patient, in accordance with a representative embodiment.

FIG. 12F is a Bland-Altman plot comparing calculated TBW and measured TBW of a leg of a patient, in accordance with a representative embodiment.

FIG. 13A is a plot of calculated intracellular volume (ICV) vs. measured ICV of an arm of a patient, in accordance with a representative embodiment.

FIG. 13B is a Bland-Altman plot comparing calculated ICV and measured ICV of an arm of a patient, in accordance with a representative embodiment.

FIG. 13C is a plot of calculated ICV vs. measured ICV of a trunk of a patient, in accordance with a representative embodiment.

FIG. 13D is a Bland-Altman plot comparing calculated ICV and measured ICV of a trunk of a patient, in accordance with a representative embodiment.

FIG. 13E is a plot of calculated ICV vs. measured ICV of a leg of a patient, in accordance with a representative embodiment.

FIG. 13F is a Bland-Altman plot comparing calculated ICV and measured ICV of a leg of a patient, in accordance with a representative embodiment.

FIG. 14A is a plot of measured ultrafiltration volume (UFV) vs. fluid overload (FO) of a leg of a patient, in accordance with a representative embodiment.

FIG. 14B is a Bland-Altman plot comparing measured UFV and FO of a leg of a patient, in accordance with a representative embodiment.

FIG. 15A is a plot of measured UFV vs. calculated UFV, in accordance with a representative embodiment.

FIG. 15B is a Bland-Altman plot comparing measured UFV and calculated UFV of a leg of a patient, in accordance with a representative embodiment.

FIG. 16A is a plot of calculated fluid overload (FO_{cal}) vs measured ultrafiltration volume (UFV_{Mea}), in accordance with a representative embodiment.

FIG. 16B is a Bland-Altman plot comparing FO_{cal} and UFV_{Mea}, in accordance with a representative embodiment.

FIG. 17A is a plot of overhydration (OH) determined using a body composition monitor (BCM) model vs. OH determined using a ratio of extracellular volume to total body water (ECV/TBW) model with a bioimpedance device, in accordance with a representative embodiment.

FIG. 17B is a Bland-Altman plot comparing OH using a BCM model and OH using a ECV/TBW model with a bioimpedance device, in accordance with a representative embodiment.

FIG. 17C is a plot of OH determined using a BCM model vs. OH determined using a ECV/TBW model with a bioimpedance device, in accordance with a representative embodiment.

FIG. 17D is a Bland-Altman plot comparing OH using a BCM model and OH using a ECV/TBW model with a bioimpedance device, in accordance with a representative embodiment.

FIG. 18 illustrates a system for fluid status determination using bioimpedance, in accordance with a representative embodiment.

FIG. 19 is a flow chart of a method of determining fluid status of a patient, in accordance with a representative embodiment.

FIG. 20 is a flow chart of a method of providing fluid status information for patients, in accordance with a representative embodiment.

FIG. 21 is a flow chart of a method of determining fluid status of a patient, in accordance with a representative embodiment.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “about,” “approximately,” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms unless specifically stated to the contrary.

The present teachings generally include devices, systems, methods, and computer-program products for determining a fluid status of patients using bioimpedance measurements and the like. For example, aspects of the present teachings include techniques to estimate fluid overload based on measurements from an eight-point electrode bioimpedance device including two (wired or wireless) handles with integrated sensors to measure distance, e.g., so that body height and segmental length can be measured and used for the estimate. In aspects, fluid overload can be estimated according to measurements of the resistance in the legs of a patient with a regression model. Aspects can also provide overhydration information according to a model from a Body Composition Monitor (BCM, produced by Fresenius Medical Care AG & Co. KGaA), and more specifically using a value of ECV/TBW measured by an eight-point electrode bioimpedance device. Further, the present teachings may include functionality to transfer data by a user device (e.g., a smartphone) to usefully manage fluid status at home by patients, e.g., dialysis patients such as peritoneal dialysis (PD) and/or hemodialysis (HD) patients.

FIG. 1 shows a patient on a footplate of a bioimpedance device in relation to a method of determining fluid status of the patient, in accordance with a representative embodiment. That is, FIG. 1 is a schematic diagram relating to a method of determining fluid status of the patient 100, specifically an assessment of hydration in the legs of the patient 100. This method may utilize a bioimpedance measurement device (otherwise referred to herein as a bioimpedance device) including a footplate 120 (e.g., a footplate including four foot electrodes, labeled E₁, E₂, E₃, and E₄ as shown). The bioimpedance device may also include a scale, where bioimpedance and body weight may automatically be measured when the patient 100 stands on the footplate 120, and/or when the footplate 120 is otherwise activated. By way of example, FIG. 1 illustrates a two-resistance model measured with foot-to-foot electrodes. Since the resistance in the right leg, R_{LR}, and the resistance in the left leg, R_{LL}, are much larger than the resistance across the lower abdomen, in certain aspects, the model can be simplified to consider only the leg resistance. Fluid overload in the legs of the patient, FO_{L}, may be calculated by the following method as described below.

The hydration factor for the patient 100, α_{P}, may be calculated by dividing a measured leg resistance R_{L} (for example, R_{LR} or R_{LL} as shown in FIG. 1) by the height (H) of the patient, as shown in Eq. 1 below.

$${α}_{P} = {R}_{L} / H \qquad Eq . 1$$

A given baseline “normal” hydration factor, α_{N}, may be determined in relation to a plurality of individuals (for example, obtained from a study of a group of healthy subjects). The difference in the hydration coefficient for the individual patient, e.g., hydration factor delta, Δα, may be calculated as shown in Eq. 2 below.

$$Δ α = {α}_{N} - {α}_{P} \qquad Eq . 2$$

Fluid overload in the legs, FO_{L}, may be calculated as shown in Eq. 3 below by multiplying the hydration factor delta, Δα, (e.g., in units of Ω/cm), by a fluid volume in legs of the patient, V_{L} (e.g., in units of liters (L)).

$$F {O}_{L} = Δα * {V}_{L} \qquad Eq . 3$$

In some aspects, the calculation of the fluid overload in legs of the patient, FO_{L}, may be further refined by multiplying Δα by V_{L} and a coefficient constant, c, where the coefficient constant, c, is determined by a calibration value from a ratio (cm/Ω).

Fluid volume in the legs of the patient, V_{L}, may be calculated as shown in Eq. 4 below, where C_{L} (Ω*cm²) is a constant coefficient (which may be determined by a study as discussed elsewhere herein).

$${V}_{L} = {C}_{L} * H / {R}_{L} \qquad Eq . 4$$

Whole body fluid overload (FO_{W}, e.g., in units of liters (L)) may be correlated with fluid overload in the legs, FO_{L}, according to Eq. 5 below, where FO_{N} is a baseline “normal” fluid status, FO_{L} is calculated as described with reference to Eq. 3, λ is a constant coefficient (liter*m²/kg) related to the body hydration excluding the legs, and BMI is a measured body mass index (kg/m²) for the patient (which may be obtained, e.g. at least in part, using sensors of the footplate 120).

$${FO}_{W} = {FO}_{N} + F {O}_{L} + λ * BMI \qquad Eq . 5$$

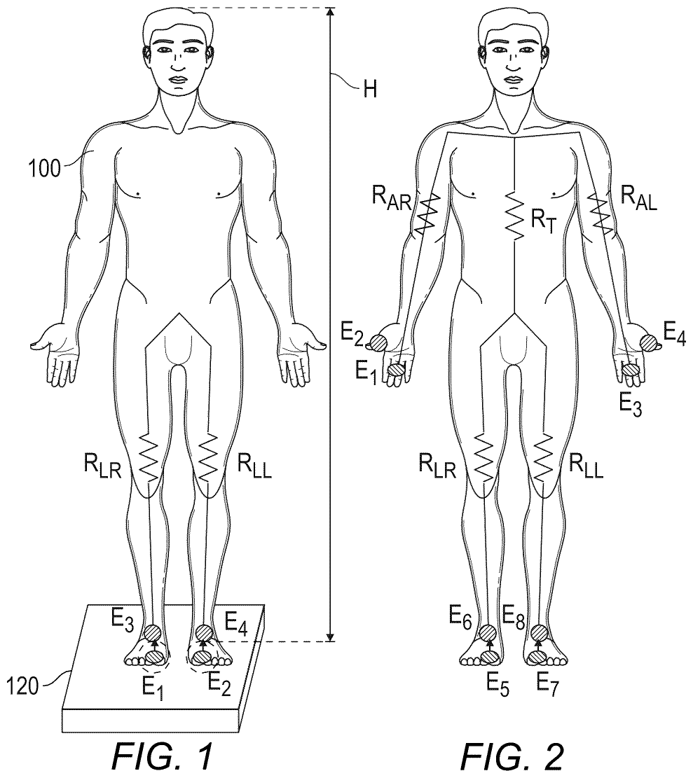

FIG. 2 is a schematic representation of resistances of a patient in relation to a method of determining fluid status of the patient. It will be understood that electrodes of a bioimpedance device are shown as E₁, E₂, E₃, E₄, E₅, E₆, E₇, E₈, as further described below, and that resistances of a patient's body segments are shown as R, with subscripts that identify the body segment (A=arm, T=trunk, and L=leg) with designations of location as right (R) and left (L), where it will be understood that these may be genericized herein to be a first side (1) and a second side (2). This labeling is provided herein by way of convenience, and other labeling is also or instead possible, as will be understood by skilled artisans.

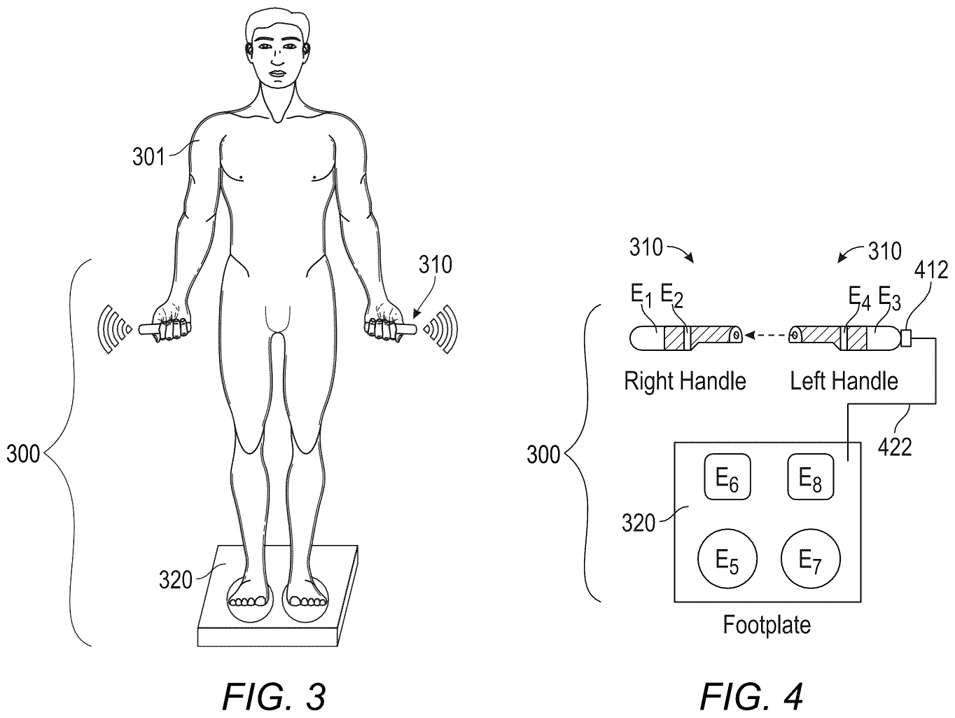

FIG. 3 shows a patient positioned for use of a bioimpedance device; and FIG. 4 shows handles and a footplate of a bioimpedance device, in accordance with a representative embodiment. The bioimpedance device 300 may include a plurality of handles 310, such as a first handle and a second handle (e.g., left handle and right handle). Each of the first handle and the second handle may include at least two electrodes, such as a metal conductor, (e.g., electrodes E₁, E₂ shown in the right handle of FIG. 4; and electrodes E₃, E₄ shown in the left handle of FIG. 4). Each of the electrodes may be configured to measure bioimpedance of a patient holding the handle 310 in which the electrode is located. The footplate 320 may include at least four electrodes (e.g., E₅, E₆, E₇ and E₈ shown in FIG. 4) configured to measure bioimpedance of the patient 301 standing on the footplate 320.

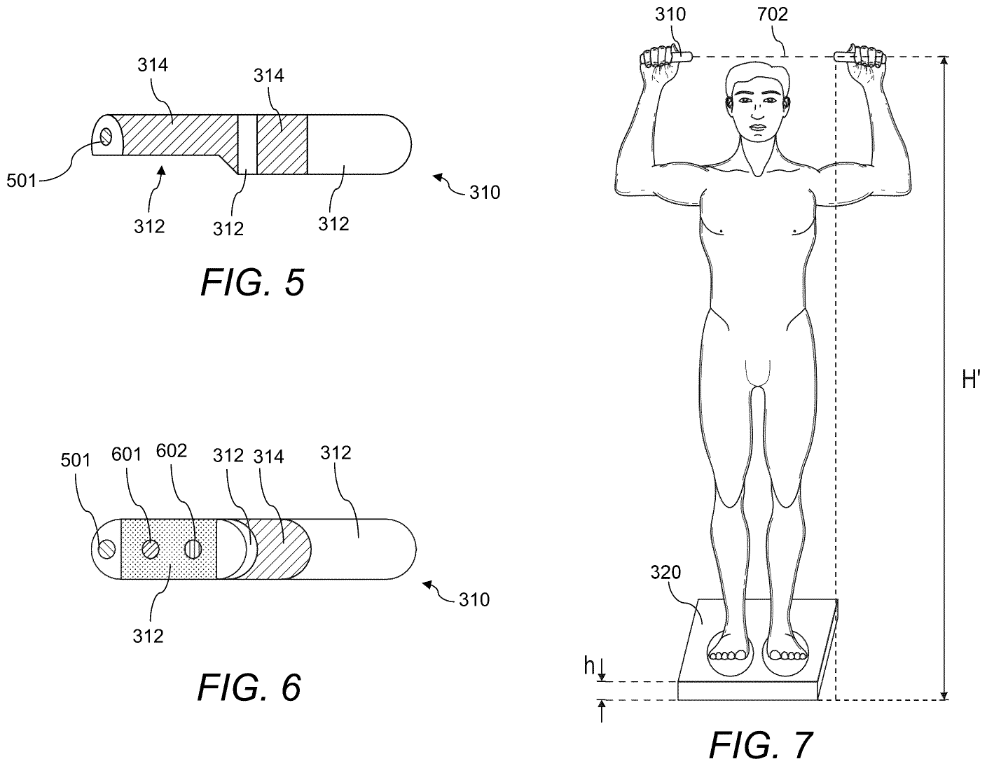

FIGS. 5 and 6 illustrate perspective and bottom plan views, respectively, of a handle 310, in accordance with a representative embodiment. The handle 310 may include one or more sensors disposed therein or thereon (e.g., integrated infrared sensors such as sensor 501, sensor 601, and sensor 602, although other sensors are also or instead possible). The handles 310 may further include one or more conductors 312 and one or more insulators 314, such that the handles 310 can provide surface electrodes that come into contact with the body and deliver the electrical current used to measure impedance.

FIG. 7 shows a patient using a bioimpedance device, in accordance with a representative embodiment (e.g., the bioimpedance device 300 described above), where the sensors of the right side handle and the left side handle may be configured to determine the height, H, of the patient (where H=H′−h in FIG. 7 as explained below; see also FIG. 1). As discussed herein, measurement of height H may be used for the calculation of fluid volume (e.g., FO_{L}, as described in Eq. 3-4 above). In the shown configuration, each of the right side handle and the left side handle may have some or all of the sensors shown in the configuration of FIGS. 5 and 6, for example. By way of example, sensor 501 of the right side handle may communicate with sensor 501 (or another sensor) in the left side handle (as illustrated by the dashed line 702 adjacent to the top of the patient's head in FIG. 7). To determine the height of the patient, the patient may grip a first handle (e.g., the right side handle) in a first hand, and the patient may grip a second handle (e.g., the left side handle) in a second hand. The patient may move each hand slowly up over their head until the infrared beam from one handle is detected by the sensor of the other handle, thereby identifying the highest point of the skull (in other words, one handle may transmit an infrared beam which may be detected by a sensor of the other handle at a point immediately adjacent to the top of the patient's head). Similarly, sensors 601 and 602 in either or both handles 310 may allow measurement of the distance, H′, between the handle(s) and floor. The height of the patient, H, can be obtained by Eq. 6, where h is the height of the footplate 320.

$$H = H ’ - h \qquad Eq . 6$$

The handles 310 may be configured for wireless communication with one another, and/or with the footplate 320. For example, the measured value of H′ may be communicated from one of the handles 310 to the footplate 320 via a wireless communication technique.

Thus, the present teachings may include an improved eight-point bioimpedance device 300 that includes one or more of: sensors configured to assist in measuring a patient's height and/or performing other functions; wireless communication capability; and/or wired connections. In this manner, while current eight-point devices may use two handles with cables to provide hand electrodes only, the present teaching may include wireless sensors (e.g., infrared and/or Bluetooth or other near-field communications technology) integrated into the handles 310 to send and receive signals from the handles 310. In an aspect, each handle 310 can communicate to another handle 310 and/or to the footplate 320 (via wireless communication or the like), e.g., when the subject's height is measured and/or for otherwise transferring data from the handles 310 to the footplate 320. Wired connections may also or instead be provided—e.g., a wire 422 providing a wired connection as shown in FIG. 4 between the handles 310 and the footplate 320, which may be included when a whole body measurement is performed. An example of wired and wireless connections is shown in Table 1 below.

TABLE 1

| | RH- | RH- | LH- | LH- | Arm- | Leg- | |
|---|---|---|---|---|---|---|---|
| | RF | LF | LF | RF | Arm | Leg | Height |
| Right | X | X | | | X | ~ | X |
| Handle | | | | | | | |
| Left | | | X | X | X | ~ | X |
| Handle | | | | | | | |
| Footplate | X | X | X | X | ~ | ~ | ~ |

In Table 1, X and ˜ represent connection of two components with wired or wireless connections, respectively; and RH, LH, RF, and LF represent a right hand, left hand, right foot, and left foot, respectively.

Therefore, as described herein, and as generally shown with reference to FIGS. 1-7 above, the present teachings may include a bioimpedance device 300 including a plurality of handles 310 including a first handle and a second handle, a footplate 320, and one or more sensors.

The handles 310 may each include at least two electrodes configured to measure bioimpedance of a patient holding the handles 310. One or more of the plurality of handles 310 may include circuitry configured for wireless communication with one or more of the footplate 320, another handle of the plurality of handles 310, and a computing device (e.g., a user's smartphone or other user device or external computing resources). In some aspects, each of the plurality of handles 310 includes a connection port 412 structurally configured to receive a wire 422 for passing a current therethrough during a bioimpedance measurement. By way of example, such a connection port 412 may include a magnet, e.g., where a connection end of a wire 422 similarly includes a magnet for ease in coupling the wire 422 to the connection port 412. Other connections are also or instead possible.

The footplate 320 may include at least four electrodes configured to measure bioimpedance of the patient standing on the footplate 320. The footplate 320 may be electrically couplable to each of the plurality of handles 310.

One or more of the sensors of the bioimpedance device 300, and more specifically, one or more of the sensors disposed on at least one of the plurality of handles 310 may be configured to determine a height of the patient as described herein. As discussed herein, one or more of the sensors may include an infrared sensor or the like, an optical sensor, a radio frequency sensor, other proximity sensors, other linear position sensors, a tracking sensor, an orientation sensor, and the like.

A technique for measuring the height of a patient will now be described, which may be the same as or similar to the technique described above. In an aspect, the height of the patient is determined by: the patient gripping a first handle in a first hand and a second handle in a second hand; the patient moving each of the first handle and the second handle from a first elevation disposed below a top of the head of the patient to a second elevation disposed immediately adjacent to the top of the head of the patient; one or more sensors of at least one of the handles 310 indicating when at least one of the first handle and the second handle is disposed at the second elevation; and measuring height of the second elevation relative to a surface using the one or more sensors. When the surface is a ground surface and the patient is standing on the footplate 320 that is positioned on the ground surface, this technique may further include subtracting a height of the footplate 320 from the height of the second elevation relative to the ground surface to determine the height of the patient.

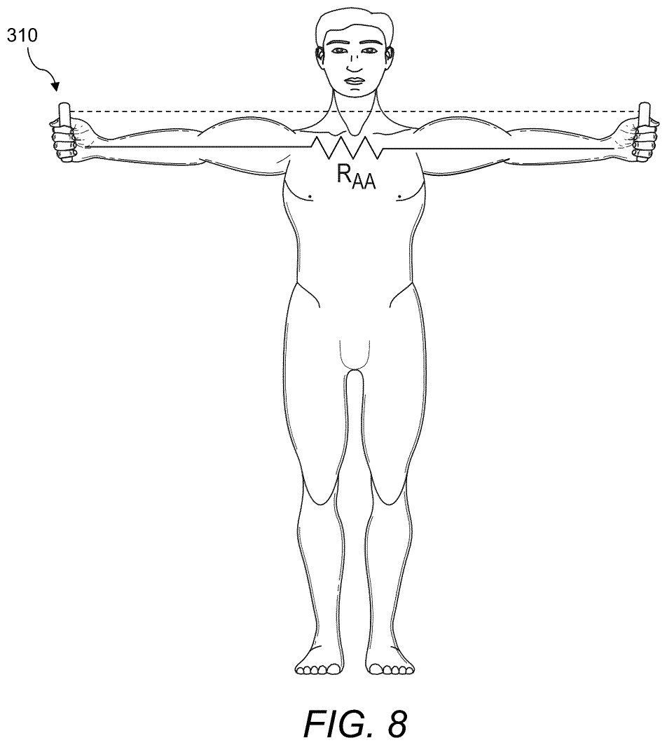

The one or more sensors of the one or more handles 310 may also or instead be configured to measure body segmental length of a patient, and/or body segmental resistance, as further described below with reference to FIG. 8.

FIG. 8 is a schematic representation of body segmental length of a patient and a resistance thereof, in accordance with a representative embodiment. Specifically, FIG. 8 illustrates a bioimpedance device including two handles 310 (e.g., which may be the same as shown and described with reference to any of FIGS. 2-7). The handles 310 may include one or more sensors configured to provide data to determine a body segmental length of the patient. FIG. 8 illustrates measurement of a body segmental length including both arms and the chest of the patient. The resistance of this segment (both arms and chest), R_{AA}, can be measured by the patient gripping the first handle in a first hand and the second handle in a second hand, and extending each hand to a furthest position possible from a torso of the patient. A distance between the first handle and the second handle may be measured, using the one or more sensors, where the distance represents the body segmental length, L. The measured resistance, R_{AA}, may be normalized over the distance as R_{AA}/L. The measured resistance, R_{AA}, is equal to the sum of resistances in both the right arm and the left arm (R_{AR}+R_{AL}) plus the resistance in the upper chest (R_{UC}). Since R_{UC} may be much smaller than the arm resistances, R_{AA} may be considered to be equal to the sum of resistance of just the arms. Assuming that the resistances in both arms are approximately identical, the resistance in each arm may be approximated as shown in Eqs. 7-9.

$${R}_{A A} = {R}_{A R} + {R}_{U C} + {R}_{A L} \qquad Eq . 7$$

$${R}_{U C} << {R}_{A R} \qquad Eq . 8$$

$${R}_{A L} = {R}_{A R} = {R}_{A A} / 2 \qquad Eq . 9$$

Stated otherwise, one or more sensors of a bioimpedance device may be configured to provide data to determine a body segmental length of the patient. For example, the body segmental length of the patient may be determined by: the patient gripping the first handle in a first hand and the second handle in a second hand; the patient extending each of the first hand and the second hand from sides of the patient at a furthest position possible from a torso of the patient; and measuring, using the one or more sensors, a distance between the first handle and the second handle, the distance representing the body segmental length. Further, the first handle and the second handle may be configured to measure a resistance across the body segmental length of the patient. The resistance may be provided to a processor configured by code stored in a memory to calculate resistance in each arm of the patient. In some aspects, the calculated resistance assumes that resistances in each arm of the patient are equal, and that resistance across the torso of the patient is negligible.

The ratio of extracellular volume (ECV) to total body water (TBW), ECV/TBW, is considered an indicator of fluid status. However, it can be difficult to use ECV/TBW to identify a degree of fluid status in clinical practice. Since the TBW includes ECV as well as intracellular volume (ICV), variation of ICV by muscle mass or fat mass could be important factors that affect accuracy of a hydration state calculated using ECV/TBW. In one aspect, fluid overload may be calculated as shown in Eq. 10 below, where coefficients β, γ, and δ are constants obtained by a multiple regression analysis.

$${FO}_{BCM} = β + γ * BMI + δ * ( ECV / TBW ) \qquad Eq . 10$$

By way of example, to obtain the coefficients β, γ and δ, a study was performed with both measurements: using a bioimpedance spectroscopy device (Hydra 4200) to provide standard fluid overload using a BCM model and by an eight-point electrode device (InBody 770 body composition analyzer) to provide ECW/TBW. A multiple regression model may be used to establish the relationship between FO_{BCM} and ECV/TBW and body mass index (BMI).

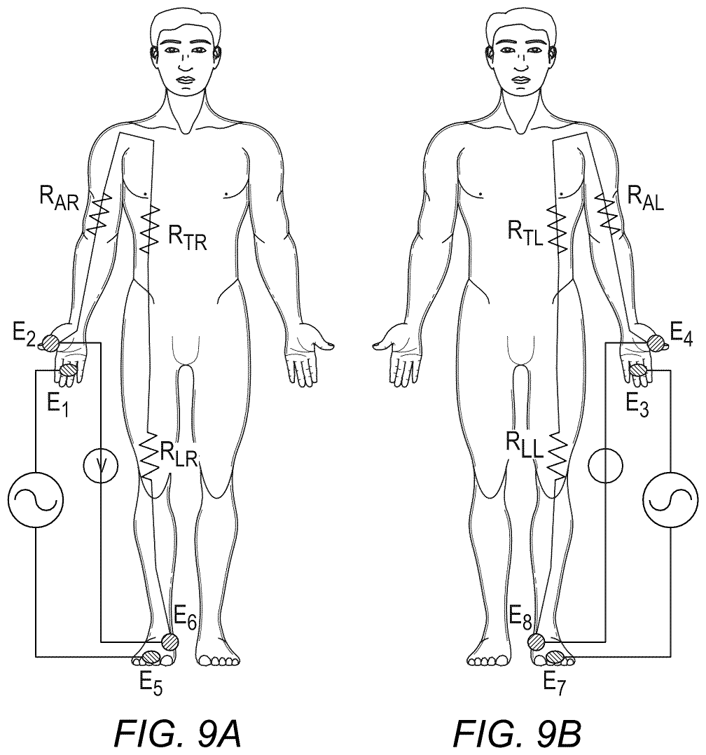

FIGS. 9A and 9B are schematic diagrams relating to a method of determining whole body bioimpedance measurement from a right side and a left side, respectively, in accordance with a representative embodiment. The following technique may be advantageous because, in many clinical applications, accurately estimating whole body resistance may be useful in addition to determining the resistance in each body segment of a patient. In this manner, measurement of segmental resistance in body segments can provide information about fluid distribution in HD patients (see Abbas S R, et al., “Effect of change in fluid distribution in segments in hemodialysis patients at different ultrafiltration rates on accuracy of whole body bioimpedance measurement,” J. Appl. Physiol., 116: 1382-1389 (2014) 10.1152/japplphysiol.01361.2013, which is hereby incorporated by reference herein). For example, knowledge of fluid distribution can improve understanding of fluid dynamics during HD. To this end, the present teachings may include a model to separately measure resistance and volume from both sides of a patient's body.

Resistance and volume may be separately measured from both body sides, and may be calculated as shown in Eq. 11 and 12 below. The whole body resistance measured in the right side, R_{WR}, may be represented as the sum of resistance in the right arm, R_{AR}, resistance in the right trunk, R_{TR}, and resistance in the right leg, R_{LR} (Eq. 11, see FIG. 9A). Similarly, the whole body resistance measured in the left side, R_{WL}, may be represented as the sum of resistance in the left arm, R_{A}, resistance in the left trunk, R_{TL}, and resistance in the left leg, R_{LL} (Eq. 12, see FIG. 9B).

$${R}_{WR} = {R}_{A R} + {R}_{T R} + {R}_{L R} \qquad Eq . 11$$

$${R}_{WL} = {R}_{A L} + {R}_{T L} + {R}_{L L} \qquad Eq . 12$$

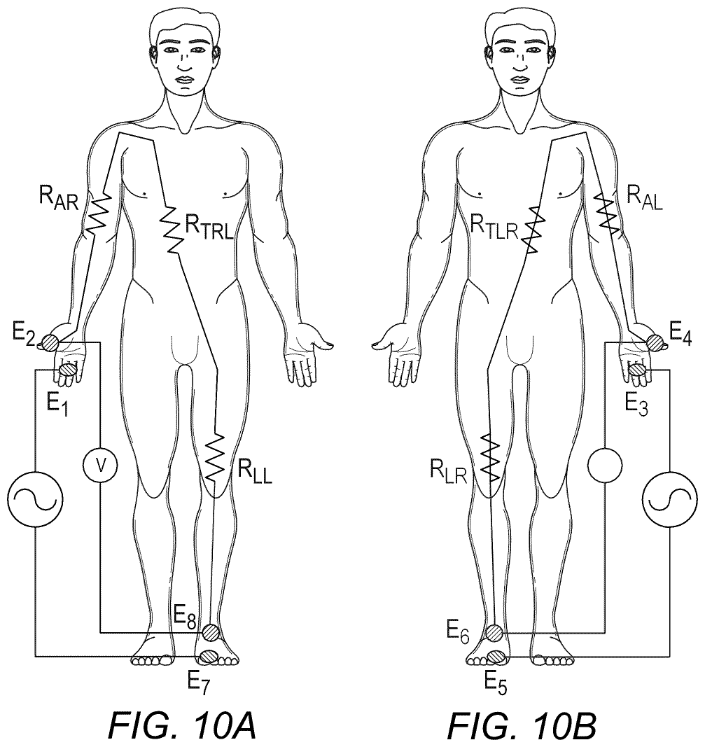

FIGS. 10A and 10B are schematic diagrams relating to a method of determining whole body bioimpedance measurement between a right arm and a left leg and between a left arm and a right leg, respectively, in accordance with a representative embodiment. The cross-body resistance from right arm to left leg, R_{WRL}, may be represented by Eq. 13 below, and cross-body resistance from left arm to right leg, R_{WLR}, may be represented by Eq. 14 below. R_{TRL} represents resistance from upper right to lower left of the trunk, and R_{TLR} represents resistance from upper left to lower right of the trunk.

$${R}_{WRL} = {R}_{A R} + {R}_{T R L} + {R}_{L L} \qquad Eq . 13$$

$${R}_{WLR} = {R}_{A L} + {R}_{T L R} + {R}_{L R} \qquad Eq . 14$$

In general, resistance in the trunk may contribute about 10% of whole body resistance (see Zhu F., et al., “Dynamics of segmental extracellular volumes during changes in body position by bioimpedance analysis,” J. Appl. Physiol., 85: 497-504 (1998) 10.1152/jappl.1998.85.2.497, which is hereby incorporated by reference), and it may be assumed that measurements of trunk resistance are approximately the same between right and left side (Eq. 15 and 16).

$${R}_{TR} = {R}_{TL} \qquad Eq . 15$$

$${R}_{TRL} = {R}_{TLR} \qquad Eq . 16$$

In Eq. 11-14, values of R_{AR} and R_{AL} may be obtained from Eq. 7 and 8. Therefore, four unknown variables R_{LR}, R_{LL}, R_{TR} (=R_{TL}) and R_{TRL} (=R_{TLR}) can be calculated by these four equations.

To calculate fluid volume with the bioimpedance method, the resistance shall be defined. Fluid volumes in extracellular (ECV) and intracellular (ICV) spaces may be obtained by: (i) the Cole model, which provides extracellular and intracellular resistances from multifrequency analyses that are used to calculate ECV and ICV (total body water (TBW) may be calculated as the sum of ECV and ICV); or (ii) with lower and higher frequency resistances, (for example, 1 kHz resistance can be used to obtain ECV and 1 MHz resistance can be used to obtain TBW), where ICV is then equal to the difference between ECV and TBW. The latter approach was used in several examples described herein. In general, the volume (V) of a cylinder with uniform electrical conductivity may be calculated using a known resistivity (p), the length (L), and a resistance (R) of the conductor, which may be determined by impedance measurement, as shown in Eq. 17 below.

$$V = ρ * {L}^{2} / R \qquad Eq . 17$$

In the human body, the arms, trunk, and legs may be considered to be cylinders, and the resistivity in extracellular fluid should be approximately the same in these different body segments so that the volume is inversely associated with resistance. However, in clinical applications, measurement of segmental length, L, can be time-consuming and inaccurate. There are known relationships between segmental length and body height (see Haapala H, et al, “Agreement Between Actual Height and Estimated Height Using Segmental Limb Lengths for Individuals with Cerebral Palsy,” Am. J. Phys. Med. Rehabil., 94: 539-546 (2015), which is incorporated by reference herein). In addition, body height may be measured more easily and accurately than the segmental length. The fluid volume, either ECV or TBW, in a specific segment Vi (where i=A, arm; i=T, trunk and i=L, leg) may therefore be represented as shown in Eq. 18 below, where Ci is the coefficient constant in segment i, H is the body height, and Ri is the resistance in the segment i. Eq. 18 may be used instead of Eq. 17, e.g., where determination of ρ and/or L² is difficult.

$$Vi = Ci * H / Ri \qquad Eq . 18$$

However, the study showed that the error of calculation with Eq. 18 can be too large for use in practical application. The error (Error) in measured volume, V_{Mea}, may be defined as shown in Eq. 19 below.

$$Error = {V}_{Mea} - {V}_{i} \qquad Eq . 19$$

The corrected volume measurement for a specific segment, V_{Mea}, may be presented as shown in Eq. 20 below.

$${V}_{Mea} = {V}_{i} + Error \qquad Eq . 20$$

It was determined in the study that the Error may be represented as a function of H, as shown in Eq. 21 below, where a and b are fitted constants and H is the height of an individual subject.

$$Error ( H ) = a * H - b \qquad Eq . 21$$

Substituting Eq. 18 and Eq. 21 into Eq. 20, the corrected volume measurement, V_{Mea}, may be calculated as a function of height, H, as shown in Eq. 22 below. An example of a process for determining parameters a, b, and Cᵢ is discussed below under Example 1.

$${V}_{Mea} = H * ( a + {C}_{i} / {R}_{i , f} ) - b \qquad Eq . 22$$

The methods described above will now be further explained by way of non-limiting examples.

### Example 1. Calculation of Segmental ECV, TBW, and ICV in the Arm, Trunk, and Leg

A total of twenty-five subjects were studied including sixteen healthy subjects (HS, age 34.8±7.6 year, sex 9F, weight 72.7±17 kg, height 168.7±9.5 cm) and thirteen hemodialysis (HD) patients (age 47±16 year, sex 4F, weight 83.9±5.4 kg, height 169.5±4.5 cm). Segmental bioimpedance data was measured with a standard eight-point bioimpedance device. Resistances in the legs were measured with four metal electrodes built into the footplate when patients were standing on the footplate (see, e.g., FIG. 1). Extracellular (1 kHz frequency) resistance (R_{L}) and volume (ECV_{L}) in the leg were collected. Body height (H), pre-HD weight (Wt) and ultrafiltration volume (UFV_{Mea}) in the HD machine were recorded. Hydration index, a, (Ω/cm) in the leg was defined as R_{L}/H in the HS (α_{H}=R_{LH}/H_{H}) and in the HD patients (α_{P}=R_{LP}/H_{P}), respectively. The difference, Δα, between the average α_{H} (̅α_{H}) in the HS and a given patient, α_{P}, (Δα=α̅_{H}−α_{P}) is indicative of the degree of FO in the legs. ECV_{2L} represents ECV on both legs. Δα*ECV_{2L} and body mass index (BMI=Wt/H²) were considered as hydration variables. Multiple regression analysis was used to find the relationship of UFV_{Mea} to FO.

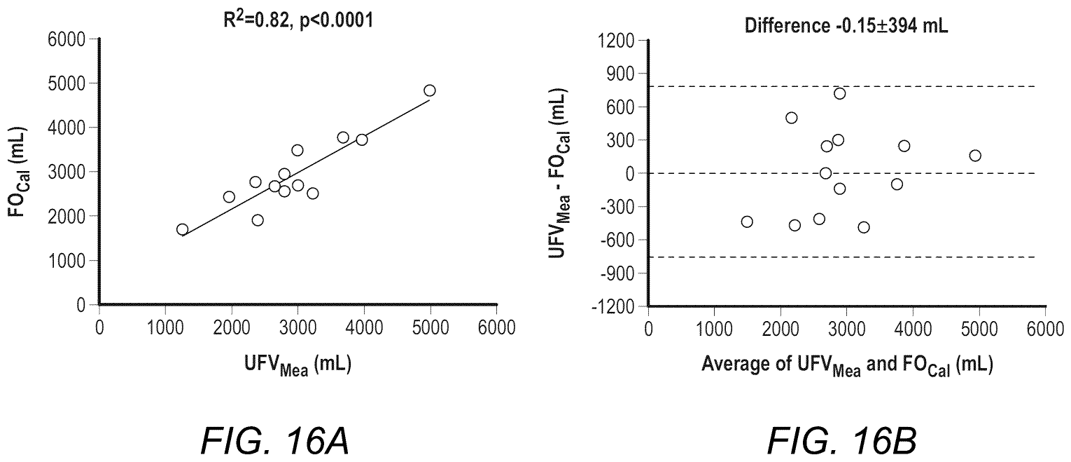

Leg resistance (R_{L}) was 217.3±55.3 (Q) pre-HD and UFV was 3862±1320 (mL). Volume in the legs (ECV_{2L}) in HD patients was significantly higher than that of HS (6.28±0.17 L vs 4.61±1.13 L, p<0.001). Average α_{H} (α̅_{H}) in the HS (1.8±0.3, Ω/cm) was considered as standard index of normal hydration in the leg. Δα was 0.24±0.37, Ω/cm and the 95% confidence interval was [0.019, 0.465]. A calculation of FO (FO_{cal}=534+0.36*Δα*ECV_{2L}+70.24*BMI, mL) was obtained. Δα and Δα*ECV_{2L} were associated with UFV_{Mea} in pre-HD measurement. FIG. 16A is a plot of calculated fluid overload (FO_{Cal}) vs measured ultrafiltration volume (UFV_{Mea}); and FIG. 16B is a Bland-Altman plot comparing FO_{Cal} and UFV_{Mea}; all in accordance with a representative embodiment. UFV_{Mea} highly correlated with FO_{Cal} (R²=0.82, p<0.0001) and Bland-Altman showed the bias with 0.15±394 mL.

The calculation of Eq. 22 was used with the HS to obtain constants a, b, and Ci (see Table 2 below). With known volumes (ECV and TBW), resistances (in 1 kHz and 1 MHz) in each segment and body height in individual HS (n=16), the parameters a, b and Ci were obtained by regression analysis.

TABLE 2
Parameters a, b, Ci for different segments

| | ECV- | ECV- | ECV- | TBW- | TBW- | TBW- |
|---|---|---|---|---|---|---|
| | Arm | Trunk | Leg | Arm | Trunk | Leg |
| a (cm²) | 0.01 | 0.08 | 0.03 | 0.02 | 0.16 | 0.06 |
| b (cm³) | 1.89 | 12.72 | 4.51 | 3.36 | 28.1 | 10.61 |
| Ci (Ω · cm²) | 1.79 | 1.02 | 4.01 | 3.5 | 1.51 | 7.89 |

The model was validated with a group of HD patients. The results indicated that ECV, TBW, and ICV in the arm and leg were estimated accurately and precisely. In this example study, the quality of measurement in the leg was most important for predicating the fluid overload (FO).

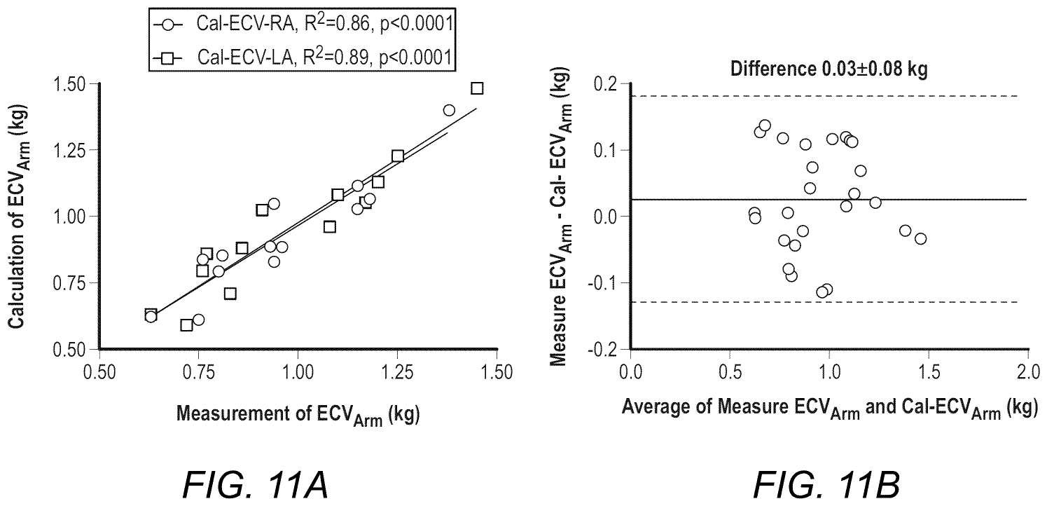

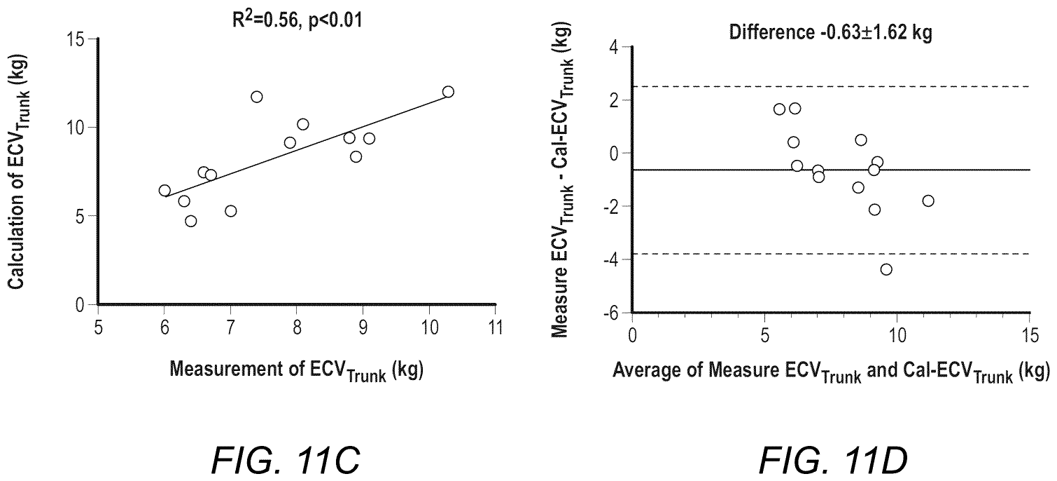

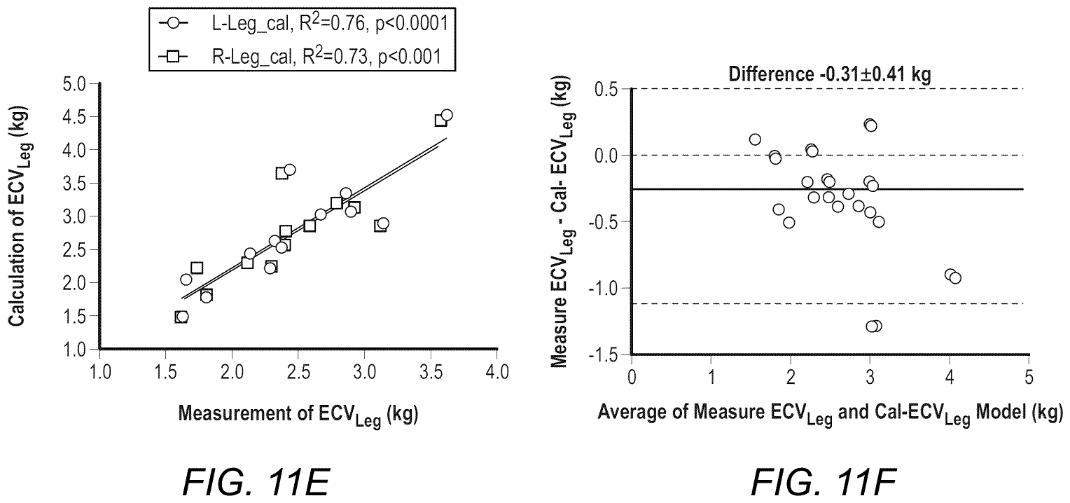

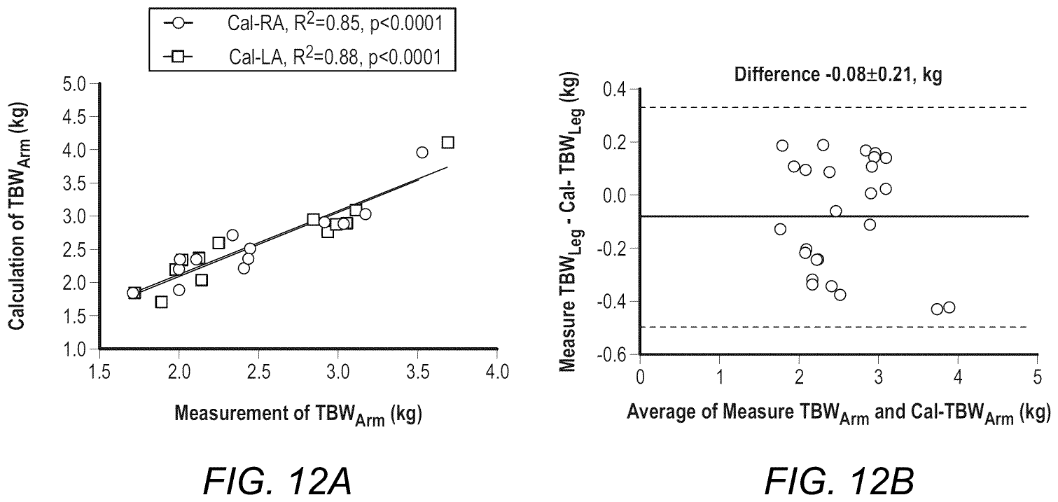

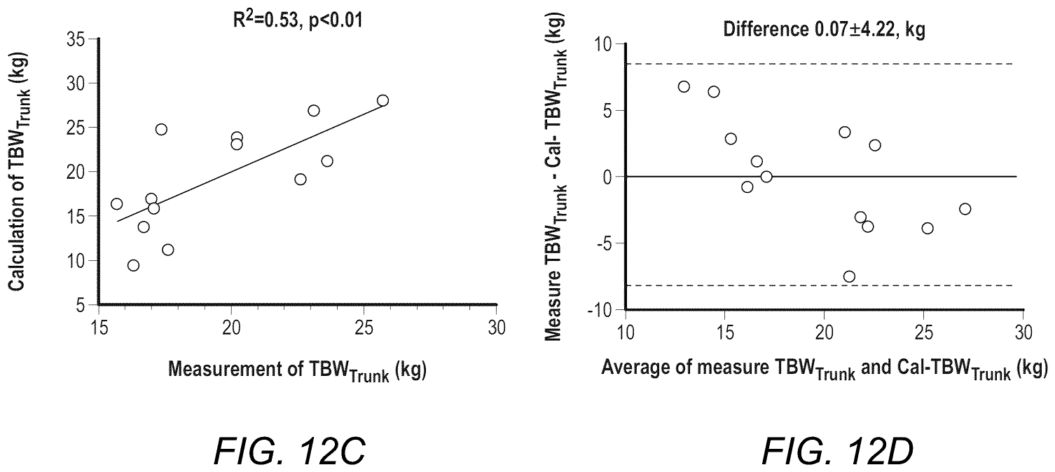

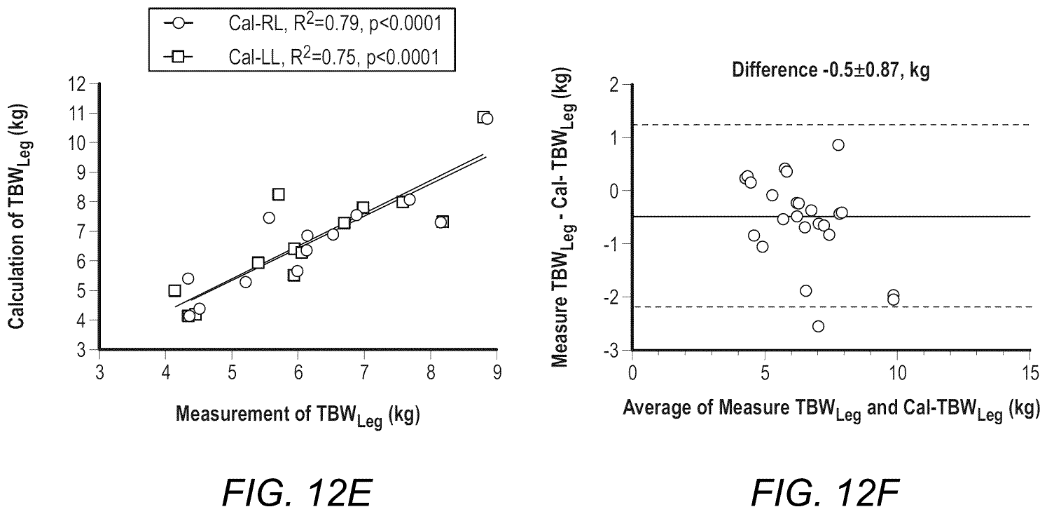

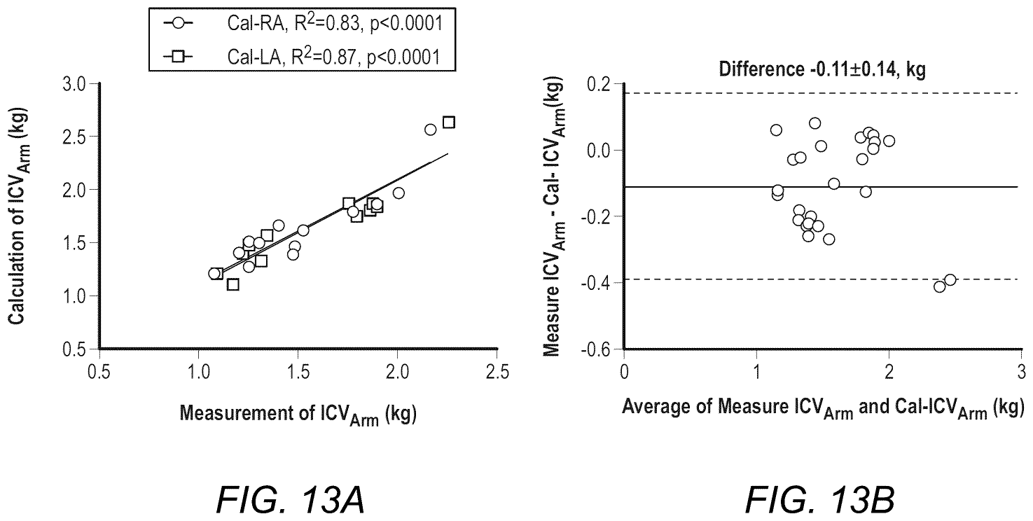

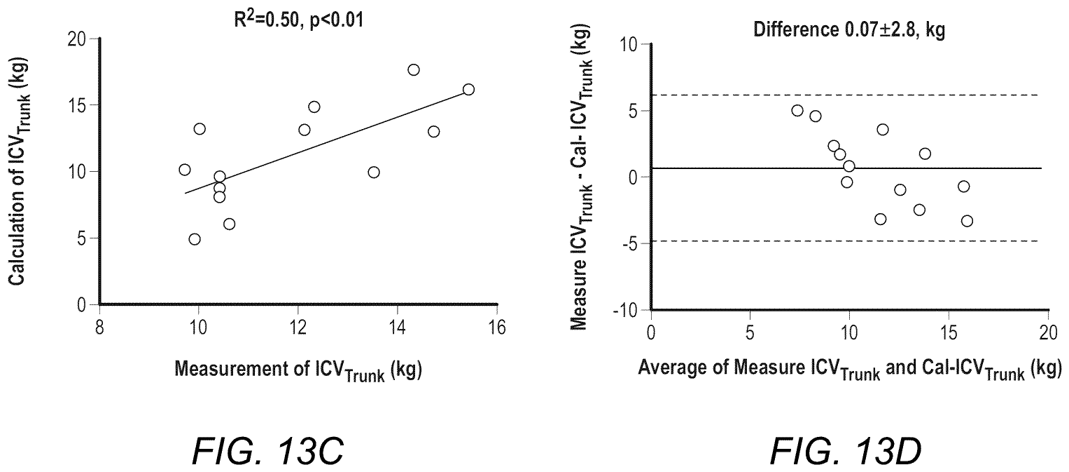

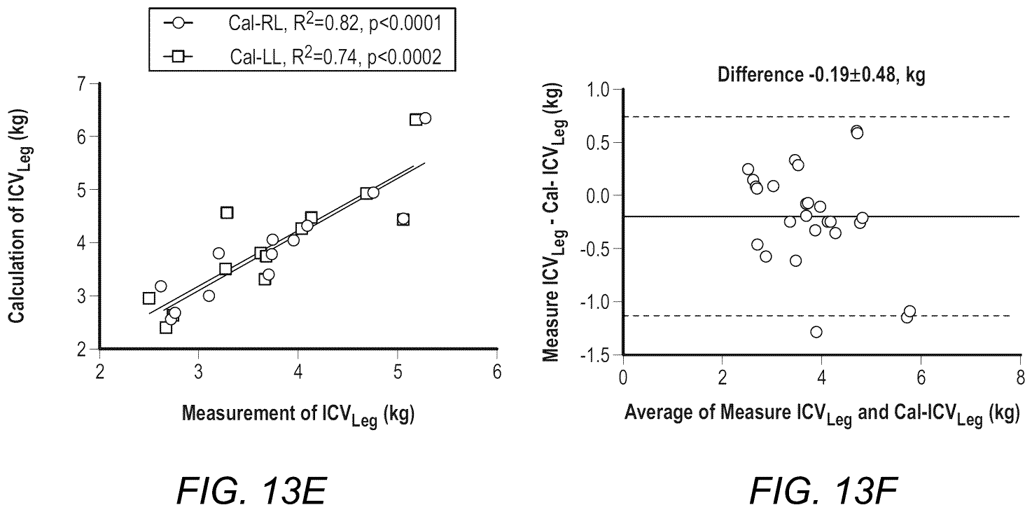

When applying the model to HD patients, ECV and TBW in each segment may be calculated with Eq. 22 according to the parameters in Table 2 and measurement of resistances at 1 kHz and 1 MHz as described herein. ICV may be calculated by the difference between ECV and TBW, when ECV and TBW are obtained with Eq. 22. Since the values of ECV, ICV, and TBW may be obtained by a standard bioimpedance device, results of the model have been validated by regression analysis and a Bland-Altman plot in HD patients, as shown in FIG. 11A-11F (ECV), FIG. 12A-12F (TBW) and FIG. 13A-13F (ICV).

FIG. 11A is a plot of calculated ECV vs. measured ECV of an arm; FIG. 11B is a Bland-Altman plot comparing calculated ECV and measured ECV of an arm; FIG. 11C is a plot of calculated ECV vs. measured ECV of a trunk; FIG. 11D is a Bland-Altman plot comparing calculated ECV and measured ECV of a trunk; FIG. 11E is a plot of calculated ECV vs. measured ECV of a leg; and FIG. 11F is a Bland-Altman plot comparing calculated ECV and measured ECV of a leg, in accordance with a representative embodiment.

FIG. 12A is a plot of calculated TBW vs. measured TBW of an arm; FIG. 12B is a Bland-Altman plot comparing calculated TBW and measured TBW of an arm; FIG. 12C is a plot of calculated TBW vs. measured TBW of a trunk; FIG. 12D is a Bland-Altman plot comparing calculated TBW and measured TBW of a trunk; FIG. 12E is a plot of calculated TBW vs. measured TBW of a leg; and FIG. 12F is a Bland-Altman plot comparing calculated TBW and measured TBW of a leg, in accordance with a representative embodiment.

FIG. 13A is a plot of calculated ICV vs. measured ICV of an arm; FIG. 13B is a Bland-Altman plot comparing calculated ICV and measured ICV of an arm; FIG. 13C is a plot of calculated ICV vs. measured ICV of a trunk; FIG. 13D is a Bland-Altman plot comparing calculated ICV and measured ICV of a trunk; FIG. 13E is a plot of calculated ICV vs. measured ICV of a leg; and FIG. 13F is a Bland-Altman plot comparing calculated ICV and measured ICV of a leg, in accordance with a representative embodiment.

These results indicate that FO estimated by the model were highly correlated with ultrafiltration volume, indicating that this method based on the calculation of the leg ECV may be useful in clinical practice.

### Example 2. Comparison of Estimation of Fluid Overload (FO) to UFV

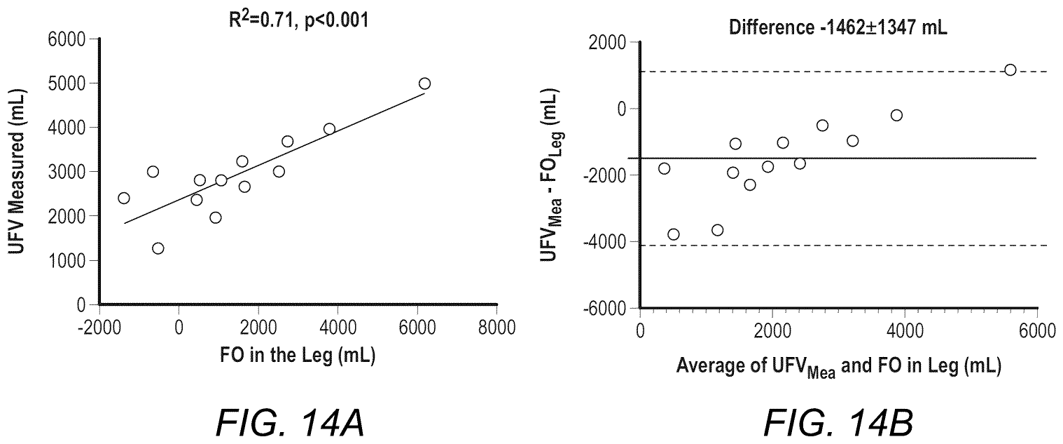

Ultrafiltration volume (UFV) may be considered comparable to fluid overload in clinical practice. In other words, UFV may be a comparator to estimate fluid overload using bioimpedance. Eq. 3 and Eq. 5 were applied in a study where a normal fluid factor (α_{N}=R_{L}/H) was determined for twelve healthy subjects, and an individual fluid factor (α_{P}=R_{L}/H) of thirteen HD patients was compared to an average of α_{N}. The difference in hydration in the leg, Δα, and FO_{L} were calculated. FIG. 14A is a plot of measured UFV vs. FO of a leg; and FIG. 14B is a Bland-Altman plot comparing measured UFV vs. FO of a leg. These results show that FO_{L} made a relatively large contribution to the ultrafiltration volume (UFV).

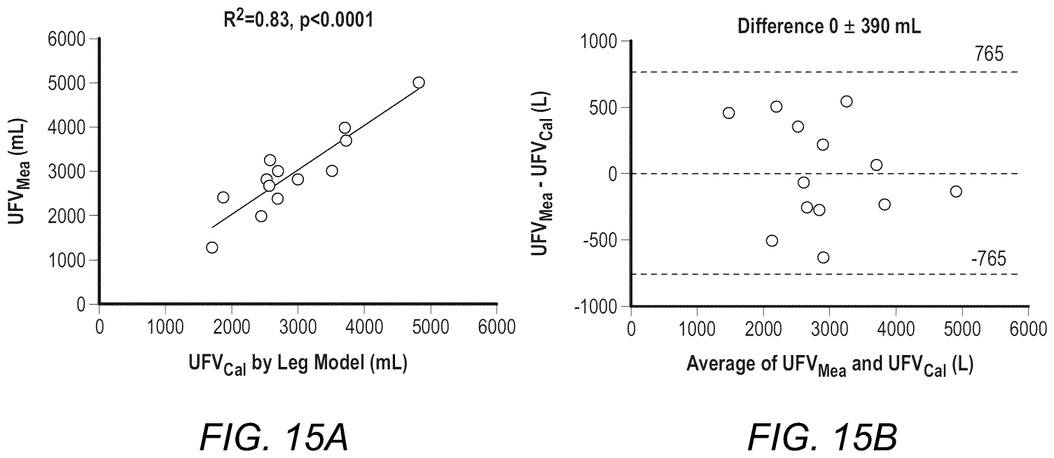

Since fluid overload in the leg (FO_{L}) should not represent total body FO_{W}, a model including FO_{L}, BMI, and a constant is provided in Eq. 23 below. FIG. 15A is a plot of measured UFV vs. UFV calculated using the model of Eq. 23; and FIG. 15B is a Bland-Altman plot comparing measured UFV and calculated UFV. FIG. 15A indicates a close correlation between the measurements and the model (R²=0.83, p<0.0001).

$${FO}_{w} = 504.2 + 364.9 * {FO}_{L} + 70.8 * BMI \qquad Eq . 23$$

### Example 3: Calculation of Overhydration with ECV/TBW Model

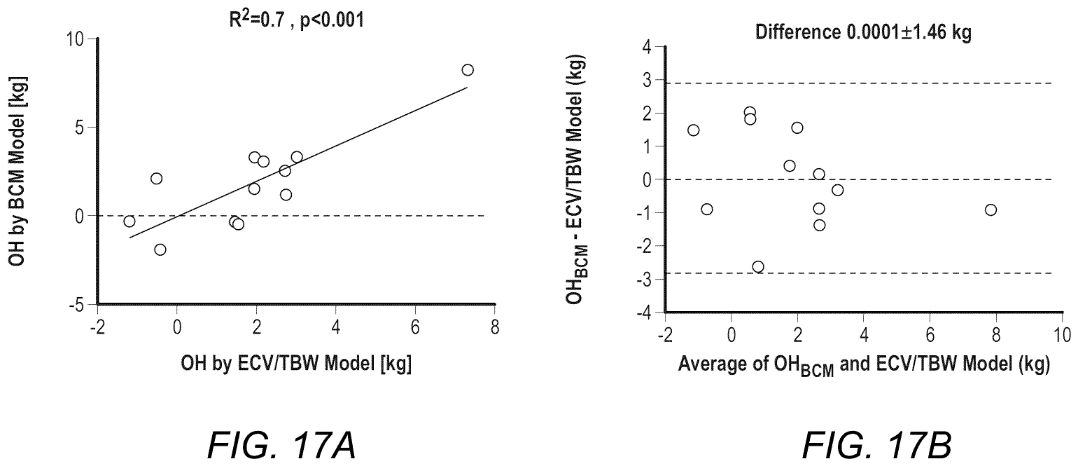

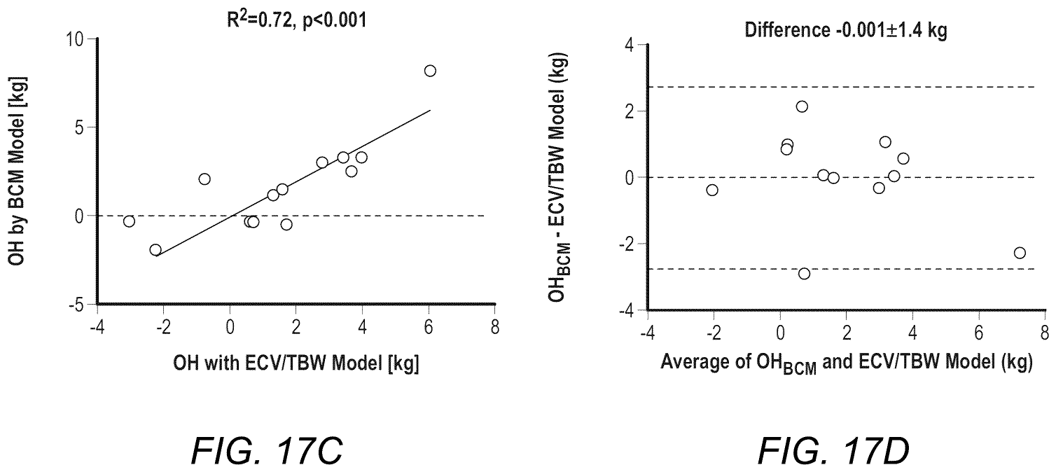

A study was performed including twelve hemodialysis (HD) patients (3 female, age 53.4±20 year, pre-HD weight 74.1±213 kg). Two eight-point bioimpedance devices were used to provide ECV/TBW values: Device 1 being InBody 770 body composition analyzer by InBody Co., LTD and Device 2 being seca mBCA 514 medical Body Composition Analyzer by seca. A Hydra 4200 bioimpedance spectroscopy device by XiTRON Technologies was used to measure whole body ECV and ICV, as well as body mass. A whole-body composition model (BCM) was applied to calculate overhydration (OH_{BCM}) based on the measurements of ECV, ICV, and BMI. Multiple regression models were established with OH_{BCM} as the dependent variable, and ECV/TBW and BMI as independent variables. The results of the two ECV/TBW models are shown in FIGS. 17A-17D.

FIG. 17A is a plot of overhydration (OH) determined using a body composition monitor (BCM) model vs. OH determined using a ratio of extracellular volume to total body water (ECV/TBW) model with a bioimpedance device; and FIG. 17B is a Bland-Altman plot comparing OH using a BCM model and OH using a ECV/TBW model with a bioimpedance device. The OH estimate by ECV/TBW model (OH₁) was obtained with eight-point bioimpedance Device 1. The multiple regression model established for Device 1 is given in Eq. 24 below.

$$O {H}_{1} = - 60.55 - 0.16 * BMI + 169.7 * ECV / TB {W}_{1} \qquad Eq . 24$$

FIG. 17C is a plot of OH determined using a BCM model vs. OH determined using a ECV/TBW model with a bioimpedance device; and FIG. 17D is a Bland-Altman plot comparing OH using a BCM model and OH using a ECV/TBW model with a bioimpedance device. The OH estimate by ECV/TBW model (OH₂) was obtained with eight-point bioimpedance Device 2. The multiple regression model established for Device 2 is given in Eq. 25 below.

$$O {H}_{2} = - 24.09 - 0.3 * BMI + 74.09 * ECV / {TBW}_{2} \qquad Eq . 25$$

These results indicate that OH based on a BCM model can be estimated based on variables of ECV/TBW and BMI in individual patients.

As discussed herein, any of the methods and techniques described herein may be performed on a system, such as a system specifically configured for fluid status determination using bioimpedance. An example of such a system is described below.

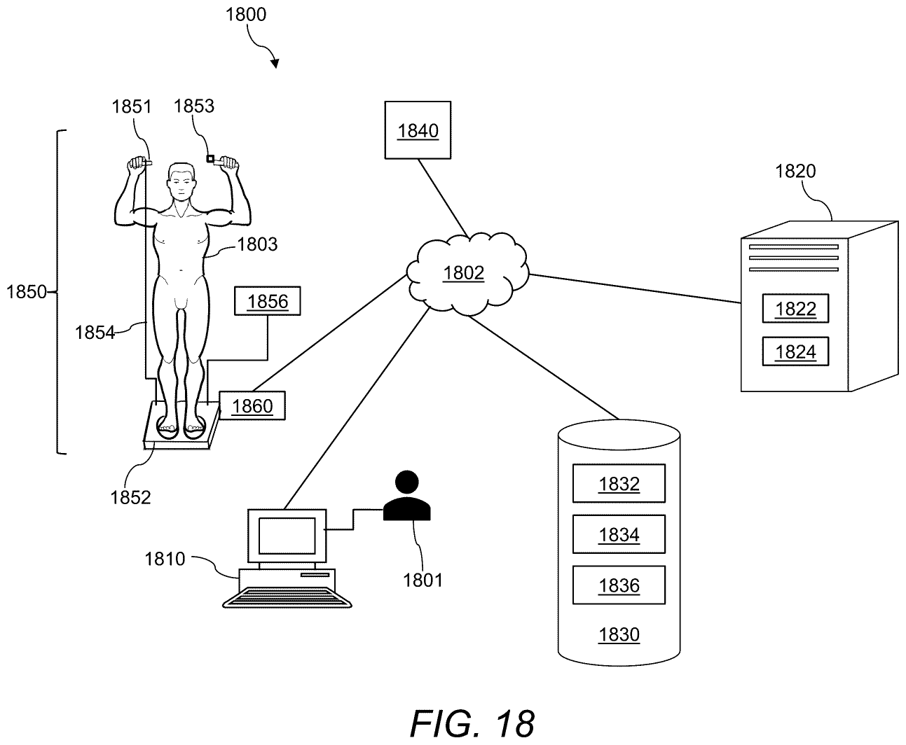

FIG. 18 illustrates a system for fluid status determination using bioimpedance, in accordance with a representative embodiment. In general, the system 1800 may include a networked environment where a data network 1802 interconnects a plurality of participating devices and/or users in a communicating relationship. The participating devices may, for example, include a bioimpedance device 1850, a user device 1810 (e.g., a computing device), a remote computing resource 1820, a database 1830, and one or more other resources 1840.

The data network 1802 may be any network(s) or internetwork(s) suitable for communicating data and information among participants in the system 1800. This may include public networks such as the Internet, private networks, telecommunications networks such as the Public Switched Telephone Network or cellular networks using third generation (e.g., 3G or IMT-2000), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 1800.

Each of the participants of the data network 1802 may include a suitable network interface comprising, e.g., a network interface card, which term is used broadly herein to include any hardware (along with software, firmware, or the like to control operation of same) suitable for establishing and maintaining wired and/or wireless communications. The network interface card may include without limitation a wired Ethernet network interface card (“NIC”), a wireless 802.11 networking card, a wireless 802.11 USB device, or other hardware for wired or wireless local area networking. The network interface may also or instead include cellular network hardware, wide area wireless network hardware or any other hardware for centralized, ad hoc, peer-to-peer, or other radio communications that might be used to connect to a network and carry data. In another aspect, the network interface may include a serial or USB port to directly connect to a local computing device such as a desktop computer that, in turn, provides more general network connectivity to the data network 1802. Thus, it will be understood that any of the components of the system may include one or more communications interfaces for communicating (e.g., wired or wirelessly transmitting and/or receiving data) with other components of the system 1800.

The bioimpedance device 1850 may be any as described herein. For example, the bioimpedance device 1850 may include one or more handles 1851 (e.g., two handles 1851), and a footplate 1852, where these components each include one or more electrodes for performing a bioimpedance operation such as using an eight point bioimpedance technique on a patient 1803. Further, one or more of these components may include one or more sensors 1853 as described herein, such as those used to measure distances and/or sense another component of the system 1800. The bioimpedance device 1850 may include connectivity 1854 between its components, such as wired and/or wireless connections between components such as the handles 1851 and the footplate 1852.

The bioimpedance device 1850 may also or instead include any component typically found in such devices or that are otherwise used to measure the impedance of biological tissue. Such components may include, for example, electrodes to apply electrical current, a current generator, a voltage amplifier, a signal processor (e.g., to process an amplified voltage signal to calculate the impedance of the tissue being measured, which can involve filtering, digitizing, and other signal processing techniques), a display (e.g., configured to display the results of a bioimpedance measurement or any other feedback described herein, including fluid status, impedance values, phase angles, and other information), a power supply, a control interface, a data output component, and the like.

The bioimpedance device 1850 may include a controller 1856, or otherwise be in communication with a controller 1856. The controller 1856 may include, or otherwise be in communication with, a processor 1822, a memory 1824, a user device 1810 such as a computing device, and so on, for controlling one or more of the components of the system 1800. Thus, in general, the controller 1856 may be electronically coupled (e.g., wired or wirelessly) in a communicating relationship with one or more of the components of a system 1800 for fluid status determination using bioimpedance and the like. The controller 1856 may also or instead be configured to adjust the frequency of the current produced by the current generator of the bioimpedance device 1850, e.g., based on signals received from a sensor 1853, or instructions received from a user 1801 or patient 1803 (who, in some aspects, may be the same individual or otherwise may be associated with one another), the processor 1822, or otherwise. In general, the controller 1856 may be electrically coupled in a communicating relationship, e.g., an electronic communication, with any of the components of the system 1800. In general, the controller 1856 may be operable to control the components of the system 1800, and may include any combination of software and/or processing circuitry suitable for controlling the various components of the system 1800 described herein including without limitation processors, microprocessors, microcontrollers, application-specific integrated circuits, programmable gate arrays, and any other digital and/or analog components, as well as combinations of the foregoing, along with inputs and outputs for transceiving control signals, power signals, sensor signals, and the like. In certain implementations, the controller 1856 may include the processor 1822 or other processing circuitry with sufficient computational power to provide related functions such as executing an operating system, providing a graphical user interface, setting and providing rules and instructions for operation of a component of the system 1800, converting sensed information into instructions, notifications, and the like, and operating a web server or otherwise hosting remote operators and/or activity through one or more communications interfaces 1860 described below. In certain implementations, the controller 1856 may include a printed circuit board, an Arduino controller or similar, a Raspberry Pi controller or the like, a prototyping board, or other computer related components.

The controller 1856 may be a local controller disposed on the bioimpedance device 1850 or another component of the system 1800, or a remote device otherwise in communication with the system 1800 and its components—e.g., the controller 1856 may be disposed on, or may include any components of, the remote computing resource 1820 as described herein, and vice-versa. For example, one or more of the controller 1856 and a user interface in communication with the controller 1856 may be disposed on an external component (e.g., a user device 1810 such as a smartphone) in communication with the system 1800 over a data network 1802.

The bioimpedance device 1850—and/or another component in the system 1800—may include one or more communications interfaces 1860 for, e.g., communication over the data network 1802, or other communication between components of the devices or systems described herein. The communications interface 1860 may include, e.g., a Wi-Fi receiver and transmitter to allow the logic calculations to be performed on a separate computing device (e.g., the user device 1810) and/or remote computing resource 1820. This may include connections to smartphone applications and the like. More generally, the communications interface 1860 may be suited such that any of the components of the system 1800 can communicate with one another. Thus, the communications interface 1860 may be present on one or more of the components of the system 1800. The communications interface 1860 may include, or be connected in a communicating relationship with, a network interface or the like. The communications interface 1860 may include any combination of hardware and software suitable for coupling the components of the system 1800 to a remote device (e.g., a computing device such as a remote computer or the like) in a communicating relationship through a data network 1802. By way of example and not limitation, this may include electronics for a wired or wireless Ethernet connection operating according to the IEEE 802.11 standard (or any variation thereof), or any other short or long range wireless networking components or the like. This may include hardware for short range data communications such as Bluetooth or an infrared transceiver, which may be used to couple into a local area network or the like that is in turn coupled to a data network such as the internet. This may also or instead include hardware/software for a WiMAX connection or a cellular network connection (using, e.g., CDMA, GSM, LTE, or any other suitable protocol or combination of protocols). Additionally, the controller 1856 may be configured to control participation by the components of the system 1800 in any network to which the communications interface 1860 is connected, such as by autonomously connecting to the data network 1802 to retrieve status updates and the like.

The user devices 1810 may include any devices within the system 1800 operated by one or more users 1801 for practicing the techniques as contemplated herein. In general, a user device 1810 may include a processor 1822 and a memory 1824, where the memory 1824 stores computer-executable code embodied in a non-transitory computer-readable medium that, when executing by the processor 1822, performs one or more steps of any method or technique described herein. Thus, although these components are shown on the remote computing resource 1820 in FIG. 18, it will be understood that processing described herein may be accomplished using one or more other devices that also or instead include a processor 1822 and a memory 1824, such as the user device 1810, the bioimpedance device 1850 (or a component thereof, such as the controller 1856), and so on. In certain aspects, the user device 1810 includes a dialysis machine and/or a component of a dialysis system. In this manner, in some aspects, the user 1801 is one or more of a dialysis patient, a dialysis technician, medical personnel such as a doctor or nurse, and so on. In other aspects, the user device 1810 is a computing device associated with one or more of a medical professional, an insurance provider or similar, a data analyst, and the like. The user devices 1810 may also or instead include any device for managing, monitoring, or otherwise interacting with tools, platforms, and devices included in the systems and techniques contemplated herein. The user devices 1810 may be coupled to the data network 1802, e.g., for interaction with one or more other participants in the system 1800.

By way of example and not limitation, the user devices 1810 may include one or more desktop computers, laptop computers, network computers, tablets, mobile devices, portable digital assistants, messaging devices, cellular phones, smart phones, portable media or entertainment devices, or any other computing devices that can participate in the system 1800 as contemplated herein. The user devices 1810 may also or instead include any form of mobile device, such as any wireless, battery-powered device, that might be used to interact with the networked system 1800. It will also be appreciated that one of the user devices 1810 may coordinate related functions (e.g., processing some or all data, storing data, etc.) as they are performed by another entity such as one of the remote computing resources 1820 or other resources 1840.

A user device 1810 may generally provide a user interface. The user interface may be maintained by a locally executing application on one of the user devices 1810 that receives data from, e.g., the remote computing resources 1820 or other resources 1840. In other embodiments, the user interface may be remotely served and presented on one of the user devices 1810, such as where a remote computing resource 1820 or other resource 1840 includes a web server that provides information through one or more web pages or the like that can be displayed within a web browser or similar client executing on one of the user devices 1810. The user interface may in general create a suitable visual presentation for user interaction on a display device of one of the user devices 1810, and provide for receiving any suitable form of user input including, e.g., input from a keyboard, mouse, touchpad, touch screen, hand gesture, or other user input device(s).

The remote computing resources 1820 may include, or otherwise be in communication with, a processor 1822 and a memory 1824, where the memory 1824 stores code executable by the processor 1822 to perform various techniques of the present teachings. More specifically, a remote computing resource 1820 may be coupled to the data network 1802 and accessible to the user device 1810 through the data network 1802, where the remote computing resource 1820 includes a processor 1822 and a memory 1824, where the memory 1824 stores code executable by the processor 1822 to perform the steps of a method according to the present teachings.

The processor 1822 may include an onboard processor for the bioimpedance device 1850 or another component of the system 1800. The processor 1822 may also or instead be disposed on a separate computing device that is connected to the system 1800 or one or more of its components through a data network 1802, e.g., using the communications interface 1860, which may include a Wi-Fi transmitter and receiver. The processor 1822 may perform calculations, e.g., calculations to determine fluid status and/or calculations related thereto (such as any as described herein), and so on.

The processor 1822 may be any as described herein or otherwise known in the art. The processor 1822 may be included on the controller 1856, or it may be separate from the controller 1856, e.g., it may be included on a user device 1810 in communication with the controller 1856 or another component of the system 1800, a remote computing resource 1820 as shown in the figure, and so on. In an implementation, the processor 1822 is included on, or is in communication with, a server that hosts an application for operating and controlling the system 1800.

The memory 1824 may be any as described herein or otherwise known in the art. The memory 1824 may contain computer code and may store data such as sequences of operation for one or more of the components of the system 1800 (e.g., the bioimpedance device 1850), sequences or content for notifications and alerts, historical data (e.g., previous inputs, measurements, and calculations), and so on. The memory 1824 may also or instead contain computer executable code stored thereon that provides instructions for the processor 1822 for implementation. The memory 1824 may include a non-transitory computer readable medium.

The remote computing resources 1820 may also or instead include data storage, a network interface (and/or other communications interface(s) 1860), and/or other processing circuitry. In the following description, where the functions or configuration of a remote computing resource 1820 are described, this is intended to include corresponding functions or configuration (e.g., by programming) of a processor 1822 of the remote computing resource 1820, or in communication with the remote computing resource 1820. In general, the remote computing resources 1820 (or processors 1822 thereof or in communication therewith) may perform a variety of processing tasks related to fluid status determination and the like as discussed herein. For example, the remote computing resources 1820 may manage information received from one or more of the user devices 1810 and/or a database 1830, and provide related supporting functions, processing one or more data sets, communicating with other resources 1840, storing data, and the like. The remote computing resources 1820 may also or instead include backend algorithms that react to actions performed by a user 1801 at one or more of the user devices 1810 and/or bioimpedance device 1850. The backend algorithms may also or instead be located elsewhere in the system 1800.

The remote computing resources 1820 may also or instead include a web server or similar front end that facilitates web-based access by the user devices 1810 to the capabilities of the remote computing resource 1820 or other components of the system 1800. A remote computing resource 1820 may also or instead communicate with other resources 1840 in order to obtain information for providing to a user 1801 and/or patient 1803 through a user interface on the user device 1810. Where the user 1801 specifies certain criteria for data processing, this information may be used by a remote computing resource 1820 (and any associated algorithms) to access other resources 1840. Additional processing may be usefully performed in this context such as recommending certain data processing operations and techniques.

A remote computing resource 1820 may also or instead maintain, or otherwise be in communication with, a database 1830 of content such as patient data 1832 related to a plurality of dialysis patients or the like, one or more constants 1834 or other pertinent information for performing any of the calculations described herein, and other data 1836 (e.g., values for calculations and/or results thereof). The database 1830 may thus be used to store any raw and/or processed/normalized data as described herein, e.g., for use by another component in the system 1800 such as the remote computing resource 1820, the bioimpedance device 1850, and/or user device 1810. In one aspect, a remote computing resource 1820 may include a database 1830 of patient data 1832, constants 1834, and other data 1836, and the remote computing resource 1820 may act as a server that provides a platform for determining fluid status using bioimpedance information obtained from the bioimpedance device 1850, providing supporting services and/or output related thereto, such as services, information, and/or recommendations related to fluid status, treatment, other support, and the like. It will be understood that the database 1830 may also or instead be independent from the remote computing resource 1820 as shown in the figure.

A remote computing resource 1820 may also or instead be configured to manage access to certain content (e.g., for an enterprise associated with a user 1801 of the user device 1810). In one aspect, a remote computing resource 1820 may manage access to a component of the system 1800 by a user device 1810 according to input from a user 1801.

The other resources 1840 may include any resources that may be usefully employed in the devices, systems, and methods as described herein. For example, the other resources 1840 may include without limitation other data networks, human actors (e.g., programmers, researchers, annotators, medical personnel, insurance providers, other medical (or medical related) support service providers, analysts, and so forth), sensors (e.g., audio or visual sensors), data mining tools, computational tools, data monitoring tools, and so forth. The other resources 1840 may also or instead include any other software or hardware resources that may be usefully employed in the networked applications as contemplated herein. For example, the other resources 1840 may include payment processing servers or platforms used to authorize payment for access, content or feature purchases, or otherwise. In another aspect, the other resources 1840 may include certificate servers or other security resources for third-party verification of identity, encryption or decryption of data, and so forth. In another aspect, the other resources 1840 may include a desktop computer or the like co-located (e.g., on the same local area network with, or directly coupled to through a serial or USB cable) with one of the user devices 1810, remote computing resources 1820, and/or bioimpedance device 1850. In this case, the other resource 1840 may provide supplemental functions for one or more of these components. Other resources 1840 may also or instead include supplemental resources such as scanners, cameras, printers, input devices, medical equipment, and so forth.

The other resources 1840 may also or instead include one or more web servers that provide web-based access to and from any of the other participants in the system 1800. While depicted as a separate network entity, it will be readily appreciated that the other resources 1840 (e.g., a web server) may also or instead be logically and/or physically associated with one of the other devices described herein, and may, for example, include or provide a user interface for web access to a remote computing resource 1820 or a database 1830 in a manner that permits user interaction through the data network 1802, e.g., from a user device 1810.

It will be understood that the participants in the system 1800 may include any hardware or software to perform various functions as described herein. For example, one or more of the user device 1810 and the other resources 1840 may include a memory 1824 and a processor 1822.

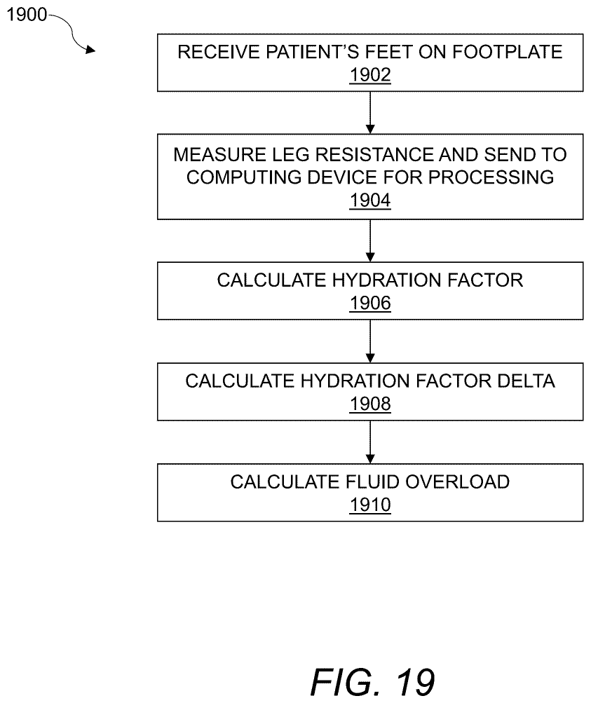

FIG. 19 is a flow chart of a method of determining fluid status of a patient, in accordance with a representative embodiment. It will be understood that the method 1900 shown and described herein (as well as the other methods described herein) may be performed using any of the devices and systems described herein, such as those described above. It will be further understood that, while the method 1900 may generally outline steps to determine the fluid status of a patient using only leg bioimpedance, the method 1900 may further include any steps of other bioimpedance techniques described herein such as a true 8-point bioimpedance technique. By way of example, one or more outputs of the method 1900—such as a calculated fluid overload, a calculated hydration factor, a calculated hydration factor delta, and the like—may be inputs of another method as described herein, and vice-versa.

As shown in step 1902, the method 1900 may include receiving a patient's feet on a footplate of a bioimpedance device. In this manner, the method 1900 may include receiving a first foot of the patient (e.g., the patient's right foot) on a footplate of a bioimpedance device, where the footplate includes a first foot electrode positioned for electrical contact with a forefoot portion of the first foot (i.e., a portion closer to the patient's toes than their heel) and a second foot electrode positioned for electrical contact with a hindfoot portion of the first foot (i.e., a portion closer to the patient's heel than their toes). And, similarly, the method 1900 may thus include receiving a second foot of the patient (e.g., the patient's left foot) on the footplate, where the footplate includes a third foot electrode positioned for electrical contact with a forefoot portion of the second foot and a fourth foot electrode positioned for electrical contact with a hindfoot portion of the second foot. Thus, in general, step 1902 may involve a patient stepping onto a footplate of a bioimpedance device (such as any as described herein) in predetermined locations for interaction with electrodes thereof for performing a bioimpedance operation and measurement.

As shown in step 1904, the method 1900 may include measuring a leg resistance, R_{L}, of the patient using the footplate by measuring resistance to a current passing through each of the first foot electrode, the second foot electrode, the third foot electrode, and the fourth foot electrode. The current may have a frequency less than or equal to 5 kilohertz. As further shown in step 1904, the method 1900 may further include providing the measured leg resistance, R_{L}, to a computing device including a processor and a memory, where the memory stores code executable by the processor to perform certain calculations using the measured leg resistance, R_{L}, such as those described below in steps 1906-1910. In some aspects, some or all parts of the computing device are included on the bioimpedance device. For example, in an aspect, the computing device is included on the footplate. In other aspects, some or all parts of the computing device are included on an external device in communication with the bioimpedance device. In general, the computing device may be any as described herein.

As shown in step 1906, the method 1900 may include calculating a hydration factor, α_{P}, by dividing the measured leg resistance, R_{L}, by height of the patient, H. In some aspects, the height of the patient is received as input provided by a user (e.g., where the user may be the patient or someone associated with the patient, such as medical personnel providing treatment for the patient). In other aspects, the height of the patient is determined, at least in part, by one or more sensors in communication with one or more of the footplate and the computing device. For example, the height of the patient may be determined using sensors included on one or more handles of the bioimpedance device using any of the techniques described herein.

As shown in step 1908, the method 1900 may include calculating a hydration factor delta, Δα, by subtracting the hydration factor, α_{P}, from a given baseline hydration factor, α_{N}. The given baseline hydration factor may be related to a plurality of individuals. For example, α_{N} may be related to data obtained from a set of healthy subjects, as described in discussion of Example 1, where α̅_{H}—the average hydration factor for a group of healthy subjects was used as a baseline hydration factor.

As shown in step 1910, the method 1900 may include calculating a fluid overload in legs of the patient, FO_{L}, by multiplying the hydration factor delta, Δα, by a fluid volume in legs of the patient, V_{L}. Several of these values are discussed in more detail below.

The fluid volume in legs of the patient, V_{L}, may be determined using the equation V_{L}=C_{L}*H/R_{L}. It will be understood that C_{L} is a constant coefficient having units Ω*cm². The constant coefficient, C_{L}, may be obtained by analyzing a plurality of subjects each having a known fluid volume in legs thereof, a known height, and a measured leg resistance. In some aspects, leg resistance of the plurality of subjects is measured using a frequency less than or equal to 5 kilohertz. An example of a method for determining C_{L} from a group of healthy subjects is discussed herein as Example 1. Example values of C_{L} are provided in Table 2.

The fluid volume in legs of the patient, V_{L}, may be determined using the equation V_{L}=H*(a+C_{L}/R_{L})−b, where C_{L} (Ω*cm²) is a constant coefficient, and constants a and b are determined from subjects to account for error as a function of H. An example of a method for determining constants a and b from a group of healthy subjects is discussed herein with respect to Example 1. Example values of a and b are provided in Table 2.

Step 1910 of the method 1900 may also or instead include calculating a whole body fluid overload, FO_{W}. For example, the method 1900 may include calculating whole body fluid overload, FO_{W}, using the equation, FO_{W}=FO_{N}+FO_{L}+λ*BMI, where FO_{N} is a baseline fluid status, FO_{L} is the calculated fluid overload in legs of the patient, λ is a constant coefficient (having units liter*m²/kg) related to the patient, and BMI is a measured body mass index (having units kg/m²) for the patient. It will be understood that the patient's BMI may be inputted by the patient or another user, and/or measured using sensors of the footplate.

An example of a method for determining FO_{N} and λ is discussed herein under Example 2. Example values of FO_{N} and k are provided in Eq. 23, where FO_{N}=504.2 L and λ=70.8 L*m²/kg.

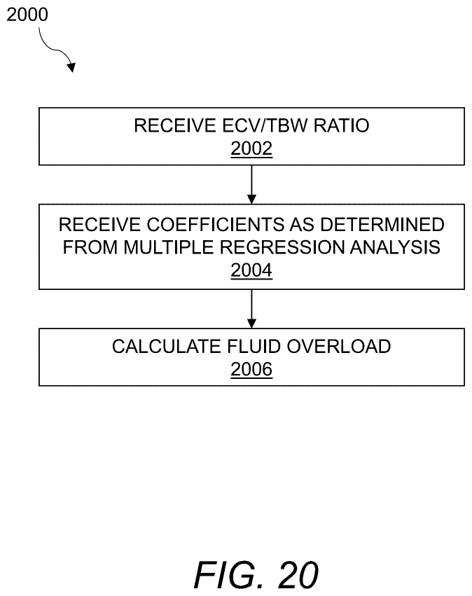

FIG. 20 is a flow chart of a method of providing fluid status information for patients, in accordance with a representative embodiment. In an embodiment, and as explained herein, the constants β, γ, and δ are obtained by a multiple regression analysis according to a known ultrafiltration volume in individual patients. It will be understood that the method 2000 shown and described herein (as well as the other methods described herein) may be performed using any of the devices and systems described herein, such as those described above. More particularly, the method 2000 shown and described herein may be performed, in whole or in part, via a computer program product for providing fluid status information for patients, the computer program product comprising computer executable code embodied in a non-transitory computer readable medium that, when executing on one or more computing devices, performs one or more of the steps of the method 2000.

In some aspects, the method 2000 may be used as a technique to improve the output of a body composition monitor (BCM), such as that produced by Fresenius Medical Care AG & Co. KGaA. In general, a BCM is a medical device that measures various parameters related to an individual's body composition, e.g., using bioimpedance spectroscopy (BIS) technology to analyze the body's resistance and reactance to a small electrical current to determine different body composition parameters. A BCM can provide information related to an individual's body fat, lean body mass, total body water, extracellular water, and the like. A BCM may be used in clinical settings, such as hospitals and dialysis centers, to monitor changes in body composition over time and assist healthcare providers make informed decisions about patient care. A BCM may be particularly useful for patients with kidney disease who undergo hemodialysis treatment, as it can help monitor changes in body water and fluid balance, which can be important factors in dialysis treatment. Additionally, a BCM can be used to monitor patients with other conditions, such as heart failure, who may have fluid retention and other changes in body composition that should be closely monitored.

As shown in step 2002, the method 2000 may include receiving a ratio (ECV/TBW) of extracellular volume, ECV, to total body water, TBW for a patient. The ratio may be provided by a body composition model using input from a bioimpedance device having at least eight electrodes.

As shown in step 2004, the method 2000 may include receiving a plurality of coefficients. The coefficients may be determined using a multiple regression analysis. The plurality of coefficients may include constants β, γ, and δ, where β is a baseline fluid overload determined by analyzing a plurality of subjects, γ is a coefficient constant having units of liter*m²*kg⁴, and δ is a coefficient constant in liters.

As shown in step 2006, the method 2000 may include calculating fluid overload (FO_{BCM}) for a patient using the formula FO_{BCM}=β+γ*BMI+δ*(ECV/TBW).

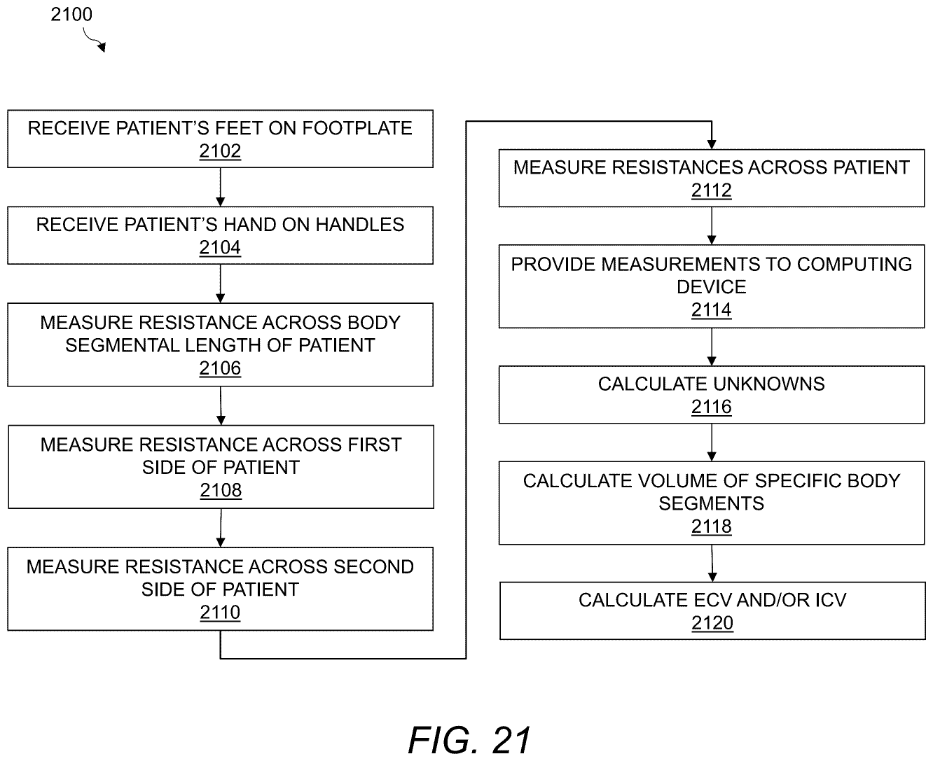

FIG. 21 is a flow chart of a method of determining fluid status of a patient, in accordance with a representative embodiment. It will be understood that the method 2100 shown and described herein (as well as the other methods described herein) may be performed using any of the devices and systems described herein, such as those described above. The method 2100 may be used to measure fluid volume by segmental resistance, where that fluid volume is then used to determine a fluid status of a patient.

As shown in step 2102, the method 2100 may include receiving a patient's feet on a footplate of a bioimpedance device—e.g., an eight point bioimpedance device—such as any as described herein. Specifically, this may include: receiving a first foot of a patient on a footplate of a bioimpedance device, the footplate including a first foot electrode positioned for electrical contact with a forefoot portion of the first foot and a second foot electrode positioned for electrical contact with a hindfoot portion of the first foot; and receiving a second foot of the patient on the footplate, the footplate including a third foot electrode positioned for electrical contact with a forefoot portion of the second foot and a fourth foot electrode positioned for electrical contact with a hindfoot portion of the second foot.

As shown in step 2104, the method 2100 may include receiving the patient's hands on handles of the bioimpedance device. Specifically, this may include: receiving a first hand of the patient (e.g., the patient's right hand) gripping a first handle of the bioimpedance device, the first handle including a first hand electrode positioned for electrical contact with a first portion of the first hand (e.g., a portion located closer to the patient's fingers than the patient's wrist) and a second hand electrode positioned for electrical contact with a second portion of the first hand (e.g., a portion located closer to the patient's wrist than the patient's fingers); and receiving a second hand of the patient (e.g., the patient's left hand) gripping a second handle of the bioimpedance device, the second handle including a third hand electrode positioned for electrical contact with a first portion of the second hand and a fourth hand electrode positioned for electrical contact with a second portion of the second hand.

As shown in step 2106, the method 2100 may include measuring a resistance, R_{A}, across a body segmental length of the patient. This may be conducted using the bioimpedance device by the patient substantially fully extending their arms away from their body while holding a handle of the bioimpedance device in each hand, and measuring a resistance to a current traversing therebetween.

As shown in step 2108, the method 2100 may include measuring a resistance, R_{W1}, across a first side of the patient. This may be conducted using the bioimpedance device—i.e., with the first foot electrode electrically coupled to the first hand electrode and with the second foot electrode electrically coupled to the second hand electrode, measuring a resistance, R_{W1}, across the first side of the patient (see, e.g., FIG. 9A and related discussion).

As shown in step 2110, the method 2100 may include measuring a resistance, R_{W2}, across a second side of the patient. This may be conducted using the bioimpedance device—i.e., with the third foot electrode electrically coupled to the third hand electrode and with the fourth foot electrode electrically coupled to the fourth hand electrode, measuring a resistance, R_{W2}, across a second side of the patient (see, e.g., FIG. 9B and related discussion).

As shown in step 2112, the method 2100 may include measuring one or more resistances across the patient. For example, this may be conducted using the bioimpedance device—i.e., with the third foot electrode electrically coupled to the first hand electrode and with the fourth foot electrode electrically coupled to the second hand electrode, measuring a resistance, R_{W12}, across the patient; and, with the first foot electrode electrically coupled to the third hand electrode and with the second foot electrode electrically coupled to the fourth hand electrode, measuring a resistance, R_{W21}, across the patient (see, e.g., FIGS. 10A and 10B and related discussion).

As shown in step 2114, the method 2100 may include providing measurements from the bioimpedance device to a computing device for processing. For example, this may include providing R_{A}, R_{W1}, R_{W2}, R_{W12}, and R_{W21} to a computing device including a processor and a memory. In some aspects, the memory stores code executable by the processor to calculate values for unknown variables by solving simultaneous linear equations. The simultaneous linear equations may include the following four equations.

Simultaneous linear equation #1: R_{W1}=R_{A1}+R_{T1}+R_{L1}, where R_{A1} represents a first arm resistance, R_{T1} represents a first side trunk resistance, and R_{L1} represents a first leg resistance.

Simultaneous linear equation #2: R_{W2}=R_{A2}+R_{T2}+R_{L2}, where R_{A2} represents a second arm resistance, R_{T2} represents a second side trunk resistance, and R_{L2} represents a second leg resistance.

Simultaneous linear equation #3: R_{W12}=R_{A1}+R_{T12}+R_{L2}, where R_{T12} represents a resistance from an upper first side to a lower second side of a trunk of the patient.

Simultaneous linear equation #4: R_{W21}=R_{A2}+R_{T21}+R_{L1}, where R_{T21} represents a resistance from an upper second side to a lower first side of the trunk of the patient.

The following may be assumed to be true in order to solve the above-identified simultaneous equations: R_{A}/2=R_{A1}=R_{A2}; R_{T1}=R_{T2}; and R_{T12}=R_{T21}.

As shown in step 2116, the method 2100 may include solving for the unknown variables (of which there are four) in the above-identified four simultaneous equations.

As shown in step 2118, the method 2100 may include calculating, via the processor, the volume of a specific body segment of the patient using the formula Vᵢ=Cᵢ*H/Rᵢ, where i represents the specific body segment, Cᵢ is a coefficient constant in segment i, H is height of the patient, and Rᵢ is a resistance measured in the segment i by the bioimpedance device. It will be understood that the patient's height, H, may be inputted by a user, sensed via the bioimpedance device, or provided by other means.

The coefficient constant in segment i, Cᵢ, may be An example of a method for determining Cᵢ from a group of healthy subjects is discussed herein as Example 1. Example values of Cᵢ for different body segments are provided in Table 2.

As shown in step 2120, the method 2100 may include calculating one or more of extracellular volume (ECV) in a specific body segment and intracellular volume (ICV) in the specific body segment.

For example, the method 2100 may further include calculating, via the processor, ECV in the specific body segment, i, of the patient using the formula ECVᵢ=C_{iE}*H/R_{iE}, where C_{iE} is an extracellular coefficient constant in segment i, H is the height of the patient, and R_{iE} is extracellular resistance measured in the segment i by the bioimpedance device.

The extracellular coefficient constant in segment i, C_{iE} (e.g., having units of liter*Ω*cm⁻¹), may be calculated by multiplying average ECV by extracellular resistance divided by body height in healthy subjects.

And for example, the method 2100 may further include calculating, via the processor, ICV in the specific body segment, i, of the patient using the formula ICVᵢ=C_{iI}*H/R_{iI}, where C_{iI} is an intracellular coefficient constant in segment i, H is the height of the patient, and R_{iI} is intracellular resistance measured in the segment i by the bioimpedance device

The intracellular coefficient constant in segment i, C_{iI} (e.g., having units of liter*Ω*cm⁻¹), may be calculated by multiplying average ECV by extracellular resistance divided by body height in healthy subjects.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from the same.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings.

Unless the context clearly requires otherwise, throughout the description, the words “comprise,” “comprising,” “include,” “including,” and the like are to be construed in an inclusive sense as opposed to an exclusive or exhaustive sense; that is to say, in a sense of “including, but not limited to.” Additionally, the words “herein,” “hereunder,” “above,” “below,” and words of similar import refer to this application as a whole and not to any particular portions of this application.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A method of determining fluid status of a patient, the method comprising:
receiving a first foot of the patient on a footplate of a bioimpedance device, the footplate including a first foot electrode positioned for electrical contact with a forefoot portion of the first foot and a second foot electrode positioned for electrical contact with a hindfoot portion of the first foot;
receiving a second foot of the patient on the footplate, the footplate including a third foot electrode positioned for electrical contact with a forefoot portion of the second foot and a fourth foot electrode positioned for electrical contact with a hindfoot portion of the second foot;
measuring a leg resistance, R_{L}, of the patient using the footplate by measuring resistance to a current passing through each of the first foot electrode, the second foot electrode, the third foot electrode, and the fourth foot electrode; and
providing the measured leg resistance, R_{L}, to a computing device including a processor and a memory, the memory storing code executable by the processor to:
calculate a hydration factor, α_{P}, by dividing the measured leg resistance, R_{L}, by height of the patient, H;
calculate a hydration factor delta, Δα, by subtracting the hydration factor, α_{P}, from a given baseline hydration factor, α_{N}, the given baseline hydration factor related to a plurality of individuals; and
calculate a fluid overload in legs of the patient, FO_{L}, by multiplying the hydration factor delta, Δα, by a fluid volume in legs of the patient, V_{L}.

2. The method of claim 1, wherein the current has a frequency less than or equal to 5 kilohertz.

3. The method of claim 1, wherein the computing device is included on the bioimpedance device.

4. The method of claim 3, wherein the computing device is included on the footplate.

5. The method of claim 1, wherein the computing device is an external device in communication with the bioimpedance device.

6. The method of claim 1, wherein the fluid volume in legs of the patient, V_{L}, is determined using the equation V_{L}=C_{L}*H/R_{L}, where C_{L} (Ω*cm²) is a constant coefficient.

7. The method of claim 6, wherein the constant coefficient, C_{L}, is obtained by analyzing a plurality of subjects each having a known fluid volume in legs thereof, a known height, and a measured leg resistance.

8. The method of claim 7, wherein leg resistance of the plurality of subjects is measured using a frequency less than or equal to 5 kilohertz.

9. The method of claim 1, wherein the fluid volume in legs of the patient, V_{L}, is determined using the equation V_{L}=H*(a+C_{L}/R_{L})−b, where C_{L} (Ω*cm²) is a constant coefficient, and constants a and b are determined from subjects to account for error as a function of H.

10. The method of claim 1, wherein the calculation of the fluid overload in legs of the patient, FO_{L}, is further refined by multiplying Δα by V_{L} and a coefficient constant, c.

11. The method of claim 10, wherein the coefficient constant, c, is determined by a calibration value from a ratio (Ω/cm).

12. The method of claim 1, wherein the memory stores code executable by the processor to calculate whole body fluid overload, FO_{W}, using the equation, FO_{W}=FO_{N}+FO_{L}+λ*BMI, where FO_{N} is a baseline fluid status, FO_{L} is the calculated fluid overload in legs of the patient, λ is a constant coefficient (L*m²/kg) related to the patient, and BMI is a measured body mass index (kg/m²) for the patient.

13. The method of claim 12, wherein FO_{N} is determined by a model fit to data from patients considered to be healthy.

14. The method of claim 12, wherein X is determined by a model fit to data from patients considered to be healthy.

15. The method of claim 12, wherein BMI is provided using sensors of the footplate.

16. The method of claim 1, wherein the height of the patient is input provided by a user.

17. The method of claim 16, wherein the user is the patient.

18. The method of claim 1, wherein the height of the patient is determined, at least in part, by one or more sensors in communication with one or more of the footplate and the computing device.

19. A computer program product for determining fluid status of a patient, the computer program product comprising computer executable code embodied in a non-transitory computer readable medium that, when executing on one or more computing devices, performs the steps of:
receiving a leg resistance, R_{L}, of a patient measured using a footplate of a bioimpedance device by measuring resistance to a current passing through each of a first foot electrode, a second foot electrode, a third foot electrode, and a fourth foot electrode;
receiving a height, H, of the patient;
calculating a hydration factor, α_{P}, by dividing the leg resistance, R_{L}, by the height of the patient;
calculating a hydration factor delta, Δα, by subtracting the hydration factor, α_{P}, from a given baseline hydration factor, α_{N}, the given baseline hydration factor related to a plurality of individuals; and
calculating a fluid overload in legs of the patient, FO_{L}, by multiplying the hydration factor delta, Δα, by a fluid volume in legs of the patient, V_{L}.

20. The computer program product of claim 19, further comprising computer executable code that, when executing on one or more computing devices, performs the step of calculating whole body fluid overload, FO_{W}, using the equation, FO_{W}=FO_{N}+FO_{L}+λ*BMI, where FO_{N} is a baseline fluid status, FO_{L} is the calculated fluid overload in legs of the patient, λ is a constant coefficient (L*m²/kg) related to the patient, and BMI is a measured body mass index (kg/m²) for the patient.
